(19) United States
(12) Patent Application Publication
(10) Pub. No.: US 2023/0211020 A1
(43) Pub. Date: Jul. 6, 2023

(54) TREATMENT OF INFECTIONS

(71) Applicant: ACT Therapeutics Ltd, Suffok (GB)

(72) Inventors: Svein KVÅLE, Ås (NO); Andrew John HEALEY, Moss (NO); Per Christian SONTUM, Oslo (NO); Spiros KOTOPOULIS, Kløfta (NO)

(73) Assignee: ACT Therapeutics Ltd, Suffolk (GB)

(21) Appl. No.: 17/928,065

(22) PCT Filed: May 27, 2021
(86) PCT No.: PCT/EP2021/064213

§ 371 Date: Nov. 28, 2022

(30) Foreign Application Priority Data
May 29, 2020 (GB) 2008094.1

(51) Int. Cl. A61K 49/22; A61K 9/00; A61K 41/00; A61K 9/107; A61P 31/04

(52) U.S. Cl. CPC A61K 49/223; A61K 9/0009; A61K 41/0028; A61K 9/1075; A61P 31/04

(57) ABSTRACT

The present invention relates to ultrasound mediated delivery of antimicrobial agents to sites of infection, and particularly for treatment of infections. Thus, the invention provides a cluster composition and a pharmaceutical composition, for use in delivery and preparation for administration of antimicrobial agents and treatment of infections.

### FIELD OF THE INVENTION

The present invention relates to ultrasound mediated delivery of antimicrobial agents to sites of infection, and particularly for treatment of infections. Thus, the invention provides a cluster composition and a pharmaceutical composition, for use in delivery and preparation for administration of antimicrobial agents and treatment of infections.

### BACKGROUND OF THE INVENTION

Antimicrobial agents are of utmost importance in modern healthcare, for treating and for preventing transmission of an ever-increasing range of infections caused by microbes such as bacteria, parasites, viruses and fungi. Antibiotics, antifungals, antivirals, and antiparasitics all have numerous and widespread uses and are prescribed and used in great quantities against various infections.

A problem with many common antimicrobial agents is their relatively low efficacy, necessitating high doses. The unutilised drug remains in the body and/or the environment, facilitating increasing drug resistance. Antimicrobial resistance is a serious global health concern, threatening our ability to treat common infectious diseases, and resulting in prolonged illness, disability, and death. Without effective antimicrobials, medical procedures such as major surgery, cancer chemotherapy, and diabetes management become very high risk. It is estimated that unless action is taken, the burden of deaths from antimicrobial resistance could balloon to 10 million lives each year by 2050, at a cumulative cost to global economic output of 100 trillion USD. Reducing the dosage of antimicrobials is thus a very important goal.

Another frequently encountered problem with such antimicrobial agents are the various side effects, ranging from diarrhoea and vomiting via headaches and fatigue to secondary infections, like yeast infections after a course of antibiotics. Toxicities of antimicrobial agents may be dose limiting, sometimes leading to a longer treatment period. Finding ways of avoiding such side effects is of importance to the healthcare system as well as to the individual patient.

Based on the above, there is a need for new and alternative compositions and methods for treatment of subjects with infections.

### BRIEF SUMMARY OF THE INVENTION

The inventors have discovered that Acoustic Cluster Therapy (ACT®) can be used to target and increase uptake of antimicrobial agents to the site of infection, thus effectively increasing efficacy and lowering toxicity through increased exposure. ACT, presented in WO2015/047103, is a concept for ultrasound mediated, targeted delivery, wherein a microbubble/microdroplet cluster composition is administered with a therapeutic agent and wherein ultrasound insonation of a targeted pathology may lead to an increase in the therapeutic effect versus using just the therapeutic agent alone. Contrasting the commonly used treatment methods and compositions with antimicrobials, which require systemic treatment with typically quite high doses even in cases where the infection is highly localised, ACT maximises clinical benefit by allowing targeted treatment as well as a higher exposure to the antimicrobial agent at the site of infection. Thus, lower doses of the antimicrobial agent may be used, limiting off-target side effects. The lowering of dosages is also financially beneficial, with the potential of considerable savings, in addition to representing an opportunity for repurposing antibiotics, ensuring drug longevity, thus addressing the problem of antimicrobial resistance. Further, the delivery speed of the antimicrobial agent can also be increased by using ACT, resulting in an increase of the period of time that the agent is present in the tissue. A wide range of drugs are time and concentration dependent, particularly compounds used to treat infections.

In one aspect, the present invention provides a pharmaceutical composition for use in a method of treatment of an infection, wherein the pharmaceutical composition comprises

(a) a cluster composition which comprises a suspension of clusters in an aqueous biocompatible medium, where said clusters have a mean diameter in the range 1 to 10 μm, and a circularity <0.9 and comprises:
(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions;
(b) an antimicrobial agent selected from the group of antibiotics, antifungals, antivirals, antiparasitics, or combinations thereof, provided as a separate composition to (a).

Hence, the invention provides a microbubble/microdroplet cluster composition for use in a method of treatment of an infection, wherein the

(a) cluster composition comprises a suspension of clusters in an aqueous biocompatible medium, where said clusters have a mean diameter in the range 1 to 10 μm, and a circularity <0.9 and comprises:
(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions;
and wherein the method comprises the administration of the (a) cluster composition and
(b) an antimicrobial agent selected from the group of antibiotics, antifungals, antivirals, antiparasitics, or combinations thereof, provided as a separate composition to (a).

Further, the invention provides a microbubble/microdroplet cluster composition described above for use in a method of delivering an antimicrobial agent or for treatment of a subject with an infection, wherein the method comprises the steps of:

(i) administering at least one antimicrobial agent selected from the group of antibiotics, antifungals, antivirals, antiparasitics, or combinations thereof to the subject;
(ii) administering the microbubble/microdroplet cluster composition to the subject; wherein the at least one antimicrobial agent is pre-, and/or co- and/or post administered to the cluster composition;
(iii) activating a phase shift of the diffusible component of the second component of the cluster composition from step (ii) by ultrasound insonation of a region of interest within said subject;
(iv) facilitating extravasation of the antimicrobial agents administered in step (i) by further ultrasound insonation of the region of interest.

In another aspect, the invention provides a system for localised delivery of an antimicrobial agent to a target location, the system comprising

(a) a cluster composition which comprises a suspension of clusters in an aqueous biocompatible medium, where said clusters have a mean diameter in the range 1 to 10 μm, and a circularity <0.9 and comprises:
(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
   where the microbubbles and microdroplets of said first and second components have opposite surface charges and form said clusters via attractive electrostatic interactions;
   (b) an antimicrobial agent selected from the group comprising antibiotics, antifungals, antivirals, antiparasitics, or combinations thereof, provided as a separate composition to (a) or together with (a).

In yet another aspect, the invention provides a method for preparing a subject for subsequent treatment with an antimicrobial agent, the method comprising the step of administering to said subject a cluster composition which comprises a suspension of clusters in an aqueous biocompatible medium, where said clusters have a mean diameter in the range 1 to 10 μm, and a circularity <0.9 and comprises:

a first component which comprises a gas microbubble and first stabiliser to stabilise said microbubble; and
a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions, the method further comprising the steps of
activating a phase shift of the diffusible component of the second component of the cluster composition by ultrasound insonation of a region of interest within said subject; and
facilitating extravasation of the antimicrobial agents that are to be administered, by further ultrasound insonation.

### BRIEF DESCRIPTION OF THE DRAWINGS

FIG. 1 provides a visualization of the two main classes of antimicrobial agents defined by their PK/PD characteristics. In both panels of FIG. 1 the curves illustrate the time (x-axis) development of the plasma concentration (y-axis) of the drug. Upper panel illustrates that for % T>MIC agents, the therapeutic efficacy of the drug is related to the fraction of time the plasma concentration of the drug is above a certain Minimum Inhibitory Concentration (MIC). Lower panel illustrates that for AUC/MIC (Area Under the Curve for drug plasma concentration) or Cmax/MIC agents, the therapeutic efficacy is related to the ratio between the AUC or Cmax and MIC.

FIG. 2 provides a visualization of cluster size versus in-vivo product efficacy, wherein the Y-axis shows the calculated correlation coefficient for Grey Scale enhancement from US imaging (i.e. amount of bubbles deposited after activation) and the X-axis shows cluster diameter in μm.

FIG. 3 provides a set up sketch of the apparatus used in Study 2 of Example 2 for application of the ACT Sonoporation procedure comprising ultrasound activation and enhancement. The numbers denote the following: 1—Amplifier, 2—Signal generator, 3—Switch box between 0.5 and 2.7 MHz, 4—Dual frequency transducer, 5—Water filled cone, 6—Water filled bag, 7—Ultrasound gel, 8—Mouse in prone position, 9—Ear bar, 10—Acoustic absorber pad.

FIG. 4 provides a box and whiskers plot of the results from the study 2 of Example 2: ACT induced delivery of nanoparticles across the Blood Brain Barrier. Upper panel: representative pictures from near infrared fluorescence (NIRF) imaging of uptake of CCPM nanoparticles to brain tissue. Control and ACT treated brains at 1 and 24 hours after ACT treatment. Lower left panel: Y-axis shows uptake as measured by NIRF as percent of injected dose per grams of brain tissue for control and ACT groups, at 1 and 24 hours after ACT treatment. Black, filled circles represent individual observations. Line and asterisk indicate statistical significance (***p<0.001) between groups derived from a t-test. Lower right panel: Y-axis shows uptake as measured by confocal microscopy as percent of brain area containing nanoparticles, for control and ACT groups, 1 hour after ACT treatment. Black, filled circles represent individual observations. Line and asterisk indicate statistical significance (*p<0.05) between groups derived from a Mann-Whitney Rank sum test.

FIG. 5 shows the dose response in the Levofloxacin treatment of thigh infection in mice. Y-axis shows the logarithm of the bacterial density in the infected tissue (Log Colony Forming Units/g) and X-axis shows Levofloxacin dose in mg/kg. Dotted line shows dose for half-maximum killing efficacy.

FIG. 6 shows the logarithm of the bacterial density in the infected tissue in an evaluation of Levofloxacin (20 mg/kg) vs. Levofloxacin (20 mg/kg)+ACT (2 ml/kg)+US treatment of thigh infection in mice. Column A shows results from Control group (saline), Column B shows results from the Levofloxacin alone group and Column C shows the results from Levofloxacin+ACT+US group.

### DETAILED DESCRIPTION OF THE INVENTION

### Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

As used herein, Acoustic Cluster Therapy (ACT), which is further defined below, comprises the administration of a cluster composition (cf. definition below) in conjunction with at least one therapeutic agent, and subsequent application of ultrasound to a targeted region of interest within a subject (e.g. infectious tissue). The term “ACT treatment”, or “ACT procedure”, is used to describe the administration and insonation of the clusters, hence including steps (iii) and (iv) in addition to the administration of the clusters.

The terms ‘treating’ and ‘treatment’ and ‘therapy’ (and grammatical variations thereof) are used herein interchangeably, and refer to 1) inhibiting the disease; for example, inhibiting a disease, condition or disorder in a subject who is experiencing or displaying the pathology or symptomatology of the disease, condition or disorder, including prevention of disease (i.e. prophylactic treatment, arresting further development of the pathology and/or symptomatology), or 2) alleviating the symptoms of the disease, or 3) ameliorating the disease; for example, ameliorating a disease, condition or disorder in an subject who is experiencing or displaying the pathology or symptomatology of the disease, condition or disorder (i.e., reversing the pathology and/or symptomatology). The treatment may relate to reducing the amount of parasites and/or arthropods. The terms may relate to the use and/or administration of medicaments, active pharmaceutical ingredients (API), and/or pharmaceutical compositions.

As used herein, the terms ‘administer’, ‘administration’, and ‘administering’ refer to (1) providing, giving, dosing and/or prescribing by either a health practitioner or their authorised agent or under their direction, a formulation, preparation or composition according to the present disclosure, and (2) putting into, taking or consuming by the subject themselves, a formulation, preparation or composition according to the present disclosure.

As used herein, ‘subject’ means any human or non-human animal selected for treatment or therapy, and encompasses, and may be limited to, ‘patient’, particularly to a human patient having an infection. None of the terms should be construed as requiring the supervision (constant or otherwise) of a medical professional (e.g., physician, nurse, nurse practitioner, physician's assistant, orderly, clinical research associate, etc.) or a scientific researcher.

The term ‘therapeutically effective amount’ as used herein means the amount of therapeutic agent (antimicrobial agent) which is effective for producing the desired therapeutic effect in a subject at a reasonable benefit/risk ratio applicable to any treatment.

The term ‘microbubble’ or ‘regular contrast microbubble’ is used in this text to describe microbubbles with a diameter in the range from 0.2 to 10 microns, typically with a mean diameter between 2 to 3 μm. ‘Regular contrast microbubbles’ include commercially available agents such as Sonazoid (GE Healthcare), Optison (GE Healthcare), Sonovue (Bracco Spa.), Definity (Lantheus Medical Imagin), and preclinical agents such as Micromarker (VisualSonics Inc.), Polyson L (Miltenyi Biotec GmbH) and Imagent® (IMCOR Pharmaceuticals Inc., San Diego, Calif., USA).

The term HEPS/PFB microbubble is used in this text to describe the microbubbles formed by reconstituting a first component (as provided in Example 1) with 2 mL of water.

The terms ‘phase shift bubbles’, ‘large, phase shift bubbles, ‘large, activated bubbles’ and ‘activated bubbles’ are used herein to describe the large (>10 μm) bubbles that form after ultrasound (US) induced activation of the cluster composition.

The term ‘microdroplet’ is used in this text to describe emulsion microdroplets with a diameter in the range from 0.2 to 10 microns.

‘Insonation’ or ‘US irradiation’ are terms used to describe exposure to, or treatment with, ultrasound.

The term “resonance frequency” or “microbubble resonance frequency”, when used in this text, is meant to describe the acoustic resonance frequency of a single bubble in an infinite matrix domain (neglecting the effects of surface tension and viscous attenuation). The resonance frequency is given by:

$$f = \frac{1}{2 π a} {( \frac{3 γ {p}_{A}}{ρ} )}^{1 / 2}$$

where a is the radius of the bubble, y is the polytropic coefficient, pA is the ambient pressure, and p is the density of the matrix.

The term ‘deposit tracer’ is used in this text in relation to the activated phase shift bubbles, in the sense that the temporary mechanical trapping of the large bubbles in the microcirculation implies that the regional deposition of phase shift bubbles in the tissue will reflect the amount of blood that flowed through the microcirculation of the tissue at the time of activated bubble deposition. Thus, the number of trapped ‘deposited’ phase shift bubbles will be linearly dependent on the tissue perfusion at the time of deposition.

The term ‘phase shift (process)’ is used in this text to describe the phase transition from the liquid to gaseous states of matter. Specifically, the transition (process) of the change of state from liquid to gas of the oil component of the microdroplets of the cluster composition.

The term ‘bi-phasic’ as used herein refers to a system comprising of two phases of state, specifically liquid and gaseous states, such as the microbubble (gas) and microdroplet (liquid) components of the cluster composition.

In this text the terms ‘therapy delivery/therapeutic agent(s)’ and ‘drug delivery/drug(s)’ are both understood to include the delivery of at least one therapeutically active agent. The therapeutic agent is for treatment of an infection and is i.e. an antimicrobial agent or a combination of two or more antimicrobial agents.

The term ‘first component’ (or 1^{st} component, or C1) is used in this text to describe the dispersed gas (microbubble) component. The term ‘second component’ (or 2^{nd} component or C2) is used in this text to describe the dispersed oil phase (microdroplet) component comprising a diffusible component.

The term ‘cluster composition’ is used in this text to describe composition resulting from a combination, such as mixing, of the first (microbubble) component and the second (microdroplet) component. Hence, the cluster composition, with characteristics as further described herein, refers to the formulated composition ready for administration to a subject, and for use in Acoustic Cluster Therapy.

The term ‘diffusible component’ is used in this text to describe a chemical component of the oil phase of the second component that is capable of diffusion in vivo into the microbubbles in the first component, transiently increasing its size.

The term ‘pharmaceutical composition’ used in this text has its conventional meaning, and in particular is in a form suitable for mammalian administration. The composition preferably comprises two separate compositions; the cluster composition (a), and the therapeutic agent (b), which are both suitable for mammalian administration such as via parenteral injection, intraperitoneal injection or intramuscular injection, either by the same or different administration routes. By the term ‘in a form suitable for mammalian administration’ is meant a composition that is sterile, pyrogen-free, lacks compounds which produce excessive toxic or adverse effects, and is formulated at a biocompatible pH (approximately pH 4.0 to 10.5). Such a composition is formulated so that precipitation does not occur on contact with biological fluids (e.g. blood), contains only biologically compatible excipients, and is preferably isotonic.

The term ‘sonometry (system)’ as used herein refers to a measurement system to size and count activated phase shift bubbles dynamically using an acoustic technique.

The term ‘reactivity’ as used herein describes the ability of the microbubbles in the first component and the microdroplets in the second component to form microbubble/microdroplet clusters upon mixing.

The terms ‘microbubble/microdroplet cluster’ or ‘cluster’ or ‘cluster composition’ as used herein refer to groups of microbubbles and microdroplets held together by electrostatic attractive forces, in a single particle, agglomerated entity. The term ‘clustering’ as used herein refers to the process where microbubbles in the first component and microdroplets of the second component form clusters.

Within medical ultrasound, acoustic power is normally described by “the Mechanical Index” (MI). This parameter is defined as the peak negative pressure in the ultrasound field (PNP) divided be the square root of the centre frequency of the ultrasound field in MHz (F_{c}) [American Institute of Ultrasound in Medicine. Acoustic Output Measurement Standard for Diagnostic Ultrasound Equipment. 1st ed. 2nd ed. Laurel, Md.: American Institute of Ultrasound in Medicine; 1998, 2003].

$$MI = \frac{PNP}{\sqrt{{F}_{c}}} .$$

Regulatory requirements during medical US imaging are to use a MI less than 1.9. During US imaging with microbubble contrast agents, an MI below 0.7 is recommended to avoid detrimental bio-effects such as micro-haemorrhage and irreversible vascular damage and using an MI below 0.4 is considered “best practise”. It should be understood that when referring to MI in the text, this reflects the in-situ MI, i.e. the MI applied to the targeted region of interest.

practice’, ref The British Medical Ultrasound Society (BMUS) Guidelines for the safe use of diagnostic ultrasound equipment 2009.

The term ‘activation’ in this text refers to the induction of a phase shift of microbubble/microdroplet clusters by ultrasound (US) irradiation.

The term “Time-dependent antimicrobial agent” or “% T>MIC” agent refers to antimicrobial agents that have a therapeutic efficacy related to the amount of time the plasma concentration of the drug is above a certain minimum level defined as Minimum Inhibitor Concentration (MIC), i.e. the minimum concentration of the drug that exerts an inhibitory effect on the microorganism to be killed.

The term “infectious disease” is an illness resulting from an infection.

The term “Concentration-dependent antimicrobial agent” or “Cmax agent” refers to antimicrobial agents that have a therapeutic efficacy related to the ratio of the maximum plasma concentration (Cmax) of the drug and MIC. This class is meant to include antimicrobial agents which are described by the alternative, PK/PD parameter describing the Area Under the Curve (AUC) of the drug plasma concentration during the dosing interval (“AUC/MIC agents”).

The term ‘antimicrobial agents’ as used herein refers to compounds (drugs, chemicals, or other substances) that either kill or slow the growth of microbes. Among the antimicrobial agents are antibacterial drugs (antibiotics), antiviral agents, antifungal agents, and antiparasitic drugs.

The term ‘infection’ as used herein refers to the invasion of a body tissue and/or organ of a subject by a disease-causing agent and/or the multiplication of said agent and/or the reaction of the host tissue/organ to said agent and/or any substances produced by it. The infection may be caused by any one or more agents from the list comprising, but not limited to, viruses, viorids, prions, bacteria, fungi, parasites, arthropods.

The term ‘site of infection’ as used herein refers to one or more sites, e.g. tissues, organs, parts of a body, wherein an infection is present. The infection may be systemic.

When referring to a specific drug, the reference is intended to include any drug comprising the same active ingredient or ingredients and with a corresponding mode of action, such as a generic drug.

### DETAILS OF THE INVENTION

Hence, delivery of an antimicrobial agent to the site of infection and treatment of an infection according to the invention can be achieved by the use of a two component, microbubble/microdroplet formulation system (i.e. the cluster composition) where microbubbles in a first component, via electrostatic attraction, are physically attached to micron sized emulsion microdroplets in a second component prior to administration. The invention uses ACT technology to generate large phase shift bubbles in vivo from an administered composition comprising microbubble/microdroplet clusters, and which facilitates delivery and uptake of separate pre-, and/or co- and/or post administered antimicrobial agent(s). When the clusters of the cluster composition are insonated with ultrasound, the volumetrically oscillating microbubbles initiate vaporisation (phase-shift) of the attached microdroplet. The enlarged resulting bubbles have been shown to deposit in capillary sized vessels in vivo and can be excited by low frequency US to induce biomechanical effects that increase drug penetration in the insonated tissue.

The composition for use in a method of treatment of an infection, according to the invention, provides improved uptake of antimicrobial agents, specifically to the site of infection, resulting in a beneficial treatment. Mixing the first component with the second component prior to administration is a pre-requisite for the efficient formation of such microbubble/microdroplet clusters. The therapeutic effect of the antimicrobial agent is considerably increased compared to administration of the agent alone, due to biomechanical mechanisms in the microvasculature, as further explained below. The present disclosure further demonstrates that a specific use of the ACT technology comprising two steps of ultrasound insonation at different frequencies and mechanical indices enables an increased delivery of separately administered antimicrobial agent(s). In one embodiment, insonation of the region of interest at a first frequency is performed in the step (iii) of activating a phase shift of the diffusible component, followed by further ultrasound insonation of the region of interest at a second lower frequency facilitating extravasation of the antimicrobial agents.

The clusters are readily activated in-vivo with low power, regular medical imaging ultrasound, i.e. with a first frequency in a range between 1-10 MHz and with a first MI of less than 1.9, such as less than 0.7, preferably less than 0.4, such as less than 0.3, which induce a liquid-to-gas transition (phase shift) of the diffusible component.

The therapeutic agent, i.e. an antimicrobial agent or a combination of two or more antimicrobial agents, is administered in conjunction with the cluster composition, i.e. pre-, and/or co- and/or post administered, and as a regular drug formulation, and in accordance with the approved route for the agent. The large, activated bubbles are temporarily retained in the microvasculature of the insonated tissue and may be utilised to facilitate drug uptake to the site of infection by further application of low frequency ultrasound, such as with a second frequency in the range of 0.2-1 MHz, most preferably between 0.4 to 0.6 MHz with an MI of 0.1-0.3. The activated phase shift bubbles are approximately 10 times larger in diameter than regular contrast microbubbles (more than 10 μm in diameter, typically 20 μm in diameter) and larger than the diameter of the microcapillaries, resulting in: trapping of the activated bubbles in the microvasculature; transient stopping of blood flow, avoiding a rapid wash out of the drug; close contact between the activated bubbles and the endothelium; orders of magnitude larger bio-effects during post activation US treatment vs. those of regular contrast microbubbles whilst still avoiding inertial cavitation mechanisms.

Hence, the invention provides a microbubble/microdroplet cluster composition for use in a method of treatment of an infection, wherein the

(a) cluster composition comprises a suspension of clusters in an aqueous biocompatible medium, where said clusters have a mean diameter in the range 1 to 10 μm, and a circularity <0.9 and comprises:
(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions;
and wherein the method comprises the administration of the (a) cluster composition, and (b) an antimicrobial agent selected from the group of antibiotics, antifungals, antivirals, antiparasitics, or combinations thereof, provided as a separate composition to (a) the cluster composition.

In such use, or method of treatment, using a microbubble/microdroplet cluster composition described above, the method comprises the steps of:

(i) administering at least one antimicrobial agent selected from the group of antibiotics, antifungals, antivirals, antiparasitics, or combinations thereof to the subject;
(ii) administering the microbubble/microdroplet cluster composition to the subject; wherein the at least one antimicrobial agent is pre-, and/or co- and/or post administered to the cluster composition;
(iii) activating a phase shift of the diffusible component of the second component of the cluster composition from step (ii) by ultrasound insonation of a region of interest within said subject;
(iv) facilitating extravasation of the antimicrobial agents administered in step (i) by further ultrasound insonation of the region of interest.

Step (iii) is in the following also referred to as the “activation step” and step (iv) is referred to as the “Enhancement Step”.

In the following sections particulars and preferred embodiments of The Cluster Composition, The First Component (microbubble), The Second Component (microdroplet), The Ultrasound Procedures, Infectious Diseases and Antimicrobial Drug(s) are disclosed and explained in further detail.

### The Cluster Composition:

The cluster composition, i.e. the combination of the first and second components, comprises clusters of gas microbubbles and oil microdroplets, i.e. is a dispersion comprising individual microbubbles and microdroplets together with stable microbubble/microdroplet clusters. ACT treatment according to the invention includes the use of a cluster formulation combining microbubbles, such as negatively charged microbubbles, with microdroplets, such as positively charged microdroplets, wherein these clusters can be activated by ultrasound. A mixture of these microbubbles and microdroplets results in small microbubble-microdroplets clusters held together by the electrostatic forces. The microdroplets typically comprise an oil component that has a boiling temperature of <50° C., and low blood solubility.

Analytical methodologies for quantitative detection and characterisation of said clusters are described in Example 1. In this text, the term ‘cluster’ refers to a group of microbubbles and microdroplets held together by electrostatic attractive forces, in a single particle, agglomerated entity. The content and size of the clusters in the cluster composition are essentially stable over some time (e.g. >1 h) after combining the first and second components in vitro, i.e. the clusters do not spontaneously disintegrate, form larger aggregates or activate (phase shifts) spontaneously, and are essentially stable over some time after dilution, even during continued agitation. It is hence possible to detect and characterise the clusters in the cluster composition with various analytical techniques that require dilution and/or agitation. Furthermore, the stability of the cluster composition allows for performing the necessary clinical procedures (e.g. reconstitution, withdrawal of dose and administration).

Each cluster in the cluster composition comprises at least one microbubble and at least one microdroplet, typically 2-20 or 2-50 individual microbubbles/microdroplets. A cluster typically has a mean diameter in the range of 1 to 10 μm and can hence flow freely in the vasculature. The clusters of the cluster composition are further characterised and separated from individual microbubbles and microdroplets by a circularity parameter. The circularity of a two-dimensional form (e.g. a projection of a microbubble, microdroplet or microbubble/microdroplet cluster) is the ratio of the perimeter of a circle with the same area as the form, divided by the actual perimeter of the form. Accordingly, a perfect circle (i.e. a two-dimensional projection of a spherical microbubble or microdroplet) has a theoretical circularity value of 1, and any other geometrical form (e.g. projection of a cluster) has a circularity of less than 1. Said clusters of the invention have a circularity <0.9. The definition of circularity parameter is further provided in WO2015/047103.

According to the invention, compositions comprising clusters with a mean size in the range of 1-10 μm, and particularly 3-10 μm, and defined by a circularity of <0.9 are considered particularly useful. In one embodiment, the mean cluster size is in the range 3-10 μm, such as 4-9 μm, such as 5-7 μm. Clusters in this size range are free-flowing in the vasculature before activation, they are readily activated by US irradiation and they produce activated bubbles that are large enough to deposit and lodge temporarily in the microvasculature. The microbubbles in the clusters permit efficient energy transfer of ultrasound energy in the diagnostic frequency range (1-10 MHz), i.e. The Activation Step (step iii)), and allows vaporisation (phase shift) of the emulsion microdroplets at low MI (under 1.9, such as under 0.7, preferably under 0.4) and diffusion of the vaporised liquid into the microbubbles and/or fusion between the vapour bubble and the microbubble. The activated bubble then expands further from the inwards diffusion of matrix gases (e.g. blood gases) to reach a volume weighted, median diameter of more than 10 μm, such as more than 15 μm, but less than 40 μm.

The formation of these clusters, i.e. by preparing a cluster composition from the first component and the second component prior to administration, is a prerequisite for an efficient phase shift event. The number and size characteristics of the clusters are strongly related to the efficacy of the composition, i.e. its ability to form large, activated (i.e. phased shifted) bubbles in vivo, and has been found to be a prerequisite for its intended functionality in vivo. The number and size characteristics can be controlled through various formulation parameters such as, but not limited to; the strength of the attractive forces between the microbubbles in the first component and the microdroplets in the second component (e.g. the difference in surface charge between the microbubbles and microdroplets): the size distribution of microbubbles and microdroplets: the ratio between microbubbles and microdroplets: and the composition of the aqueous matrix (e.g. buffer concentration, ionic strength). The mean circular equivalent diameter of the clusters formed should preferably be larger than 3 μm, more preferably between 5 to 7 μm, but smaller than 10 μm. The concentration of clusters between 3 to 10 μm in the combined preparation (cluster composition) should preferably be more than 10 million/m L, more preferably more than 20 million/m L. In one embodiment, based on the results shown in Tables 5 and 6 of WO2015/047103, the composition should comprise at least 0.6 million/ml of clusters with the mean size 5-10 μm. In another embodiment, the composition for administration should comprise at least 3 million/mL of clusters with a diameter between 5-10 um. Such a minimum would, according to FIG. 11 of applicant's WO2015047103, assure an enhancement of >150 GS units, and a certain, minimum level of product efficacy and therapeutic benefit. In another embodiment the cluster concentration of clusters in size range 1-10 μm should be at least 10 million/mL, such as at least around 25 million/ml.

The size of the activated bubbles can be engineered by varying the size distribution of the microdroplets in the emulsion and the size characteristics of the clusters (see Example 1 of WO2015/047103). The clusters are activated to produce large bubbles by application of external ultrasound energy, after administration, such as from a clinical ultrasound imaging system, under imaging control. The large phase shift bubbles produced are typically of a diameter of 10 μm or more. Low MI energy levels, which are well within the diagnostic imaging exposure limits (MI<1.9), are sufficient to activate the clusters, which makes the technology significantly different from the other phase transition technologies available (e.g. acoustic microdroplet vaporisation (ADV)). Due to their large size, the activated bubbles temporarily lodge in the microvasculature and can be spatially localised in a tissue or organ of interest, such as an infected tissue or organ, by spatially localised ultrasound insonation, i.e. insonation focused on the pathological region of interest. Hence, after administration of the cluster composition, the clusters are activated within, at or near the site of the infection by deposition of ultrasound energy towards the site of the infection. The large, activated bubbles produced (10 μm or more in diameter) have acoustic resonances at low ultrasound frequency (1 MHz or less, typically 0.5 MHz).

It will be appreciated by the person of skill in the art that for the composition for use and method and system of the invention, a further irradiation of the large activated bubbles with the application of low frequency ultrasound further enhances the uptake of the antimicrobial agent(s). The ultrasound procedures are further detailed under the heading: The Ultrasound Procedures. Hence, it has been found that e.g. the application of low frequency ultrasound during the Enhancement Step (Step iv)) can be used to produce mechanical (shear forces and microstreaming) and/or thermal bio-effect mechanisms to increase the permeability of the vasculature and/or sonoporation and hence increase delivery and retention of the antimicrobial agent to the targeted tissue. This mechanism has shown to increase vascular permeability so a higher pay load of drug can be delivered (ref WO2015047103A1). Further it can improve the distribution of drug in the infectious tissue, and it can increase the uptake of drug to pathological cells. It may also be that the bio-mechanical effects towards the endothelial cells can result in the generation of biochemical signals and the onset of immune responses that further improves the therapeutic efficacy.

If comparing the compositions and methods and systems of the invention with methods wherein free-flowing, regular contrast microbubbles are used, the large phase shift microbubbles of the current invention are entrapped in a segment of the vessels and the microbubble surface is in close contact with the endothelium. In addition, the volume of an activated bubble is typically 1000 times that of a regular microbubble. At equal MI, insonated at a frequency close to resonance for both bubble types (0.5 MHz for phase shift microbubbles and 5 MHz for regular contrast agent microbubbles), it has been shown that the absolute volume displacement during oscillations is almost three orders of magnitude larger with the phase shift bubbles than with a regular contrast microbubble. Hence, insonation of phase shift bubbles will produce completely different levels of bio-mechanical effects, with significantly larger effect size and penetration depth than during insonation of regular contrast microbubbles. The bio-effects observed with free-flowing, regular contrast microbubbles are likely dependent upon cavitation mechanisms, with ensuing safety concerns such as micro-haemorrhage and irreversible vascular damage. The larger phase shift bubbles can be oscillated in a softer manner (lower MI, e.g. <0.3), avoiding cavitation mechanisms, but still inducing sufficient mechanical work to enhance the uptake of antimicrobial agent from the vasculature and into the target tissue. The trapping of the large phase shift bubbles will also act as a deposit tracer. This further allows quantification of the number of activated clusters and perfusion of the tissue and allows contrast agent imaging of the tissue vasculature to identify the spatial extent of the pathology to be treated.

In the system or method of the invention, or in the pharmaceutical composition for use, the cluster composition may comprise either of a wide range of gases and first stabilizers for the first, microbubble component and oils and second stabilizers for the second, microdroplet component. Example 5-2 of WO2015/047103 A1 provides results from a study where a variety of first components, in terms of gases and first stabilizers, were explored. This example demonstrated that a cluster composition useful according to the invention formed for most all combinations explored. Furthermore, in Example 5-3 of the same, a variety of oils for the second component were explored. This example demonstrated that a stable cluster composition, which activated upon US insonation, formed for a range of oil components, preferably those with a water solubility less than 1·10⁻⁴ M, such as less than 1·10⁻⁵ M. Both examples are incorporated herein by reference. Furthermore, in Example 6 of the current disclosure, second components with a variety of oils are combined with first components with a variety of gases and first stabilizers. All these combinations will demonstrate that the cluster composition of the current invention may comprise either of a wide range of gases and first stabilizers for the first, microbubble component and oils and second stabilizers for the second, microdroplet component.

Hence, in a preferred embodiment, the cluster compositions comprises a first component comprising microbubbles of a perfluorated gas stabilized with a first stabilizer selected from a group of polymers, proteins, phospholipids and surfactants. Further in a preferred embodiment, the cluster compositions comprises a second component comprising microdroplets comprising a halogenated oil stabilized with a second stabilizer selected from a group of polymers, proteins, phospholipids and surfactants. Hence, in one embodiment, the cluster composition comprises the above combination of the first and second components.

In some embodiments, only the cluster composition without an antimicrobial agent is administered to a subject, for the preparation of a subject for a subsequent administration of an antimicrobial agent. In such embodiments, the administration of the cluster composition and the application of the ultrasound procedures is such that the administration is not a treatment, but a preparation for a treatment.

### The First Component (Microbubble):

The first component comprises a gas microbubble comprising a gas (“dispersed gas”) and a first stabiliser to stabilise said gas. The microbubbles may be similar to conventional ultrasound contrast agents that are on the market and approved for use for several clinical applications, such as Sonazoid, Optison, Definity or Sonovue, or similar agents used for pre-clinical application such as Micromarker and Polyson L. The first component may be in the form of an injectable aqueous dispersion or a lyophilized powder for reconstitution. Any biocompatible gas may be present in the microbubbles, the term ‘gas’ as used herein including any substance (including mixtures) at least partially, e.g. substantially or completely in gaseous (including vapour) form at the normal human body temperature of 37° C. The gas may thus, for example, comprise air; nitrogen; oxygen; carbon dioxide; hydrogen; an inert gas such as helium, argon, xenon or krypton; a sulphur fluoride such as sulphur hexafluoride, disulphur decafluoride or trifluoromethylsulphur pentafluoride; selenium hexafluoride; an optionally halogenated silane such as methylsilane or dimethylsilane; a low molecular weight hydrocarbon (e.g. containing up to 7 carbon atoms), for example an alkane such as methane, ethane, a propane, a butane or a pentane, a cycloalkane such as cyclopropane, cyclobutane or cyclopentane, an alkene such as ethylene, propene, propadiene or a butene, or an alkyne such as acetylene or propyne; an ether such as dimethyl ether; a ketone; an ester; a halogenated low molecular weight hydrocarbon (e.g. containing up to 7 carbon atoms); or a mixture of any of the foregoing. Advantageously, the gas is a halogenated gas, such as a perfluorinated gas. Advantageously at least some of the halogen atoms in halogenated gases are fluorine atoms; thus, biocompatible halogenated hydrocarbon gases may, for example, be selected from bromochlorodifluoromethane, chlorodifluoromethane, dichlorodifluoro-methane, bromotrifluoromethane, chlorotrifluoromethane, chloropenta-fluoroethane, dichlorotetrafluoroethane, chlorotrifluoroethylene, fluoroethylene, ethylfluoride, 1,1-difluoroethane and perfluorocarbons. Representative perfluorocarbons include perfluoroalkanes such as perfluoromethane, perfluoroethane, perfluoropropanes, perfluorobutanes (e.g. perfluoro-n-butane, optionally in admixture with other isomers such as perfluoro-iso-butane), perfluoropentanes, perfluorohexanes or perfluoroheptanes; perfluoroalkenes such as perfluoropropene, perfluorobutenes (e.g. perfluorobut-2-ene), perfluorobutadiene, perfluoropentenes (e.g. perfluoropent-1-ene) or perfluoro-4-methylpent-2-ene; perfluoroalkynes such as perfluorobut-2-yne; and perfluorocycloalkanes such as perfluorocyclobutane, perfluoromethylcyclobutane, perfluorodimethylcyclobutanes, perfluorotrimethyl-cyclobutanes, perfluorocyclopentane, perfluoromethyl-cyclopentane, perfluorodimethyl-cyclopentanes, perfluorocyclohexane, perfluoromethylcyclohexane or perfluorocycloheptane. Other halogenated gases include methyl chloride, fluorinated (e.g. perfluorinated) ketones such as perfluoroacetone and fluorinated (e.g. perfluorinated) ethers such as perfluorodiethyl ether.

The use of perfluorinated gases, for example sulphur hexafluoride and perfluorocarbons such as perfluoropropane, perfluorobutanes, perfluoropentanes and perfluorohexanes, are particularly advantageous in view of the recognised high stability in the bloodstream of microbubbles containing such gases. Other gases with physicochemical characteristics which cause them to form highly stable microbubbles in the bloodstream may likewise be useful. Preferably, the dispersed gas comprises sulphur hexafluoride, perfluoropropane, perfluorobutane, perfluoropentane, perflurohexane (i.e. a C3-6 perfluorocarbon), nitrogen, air or any mixture thereof, and preferably comprises sulphur hexafluoride or a C3-6 perfluorocarbon. In one embodiment, the gas of the first component is selected from the group of sulphur fluorides and halogenated low molecular weight hydrocarbons (e.g. containing up to 7 carbon atoms). In some embodiments, the dispersed gas comprises sulphur hexafluoride, perfluoropropane, or perfluorobutane, or any mixture thereof. In specific embodiments, the dispersed gas is perfluorobutane.

The dispersed gas may be in any convenient form, for example using any appropriate gas-containing ultrasound contrast agent formulation as the gas-containing component such as Sonazoid, Optison, Sonovue or Definity or pre-clinical agents such as Micromarker or PolySon L. The first component will also contain material in order to stabilise the microbubble dispersion, in this text termed ‘first stabiliser’. Representative examples of such formulations include microbubbles of gas stabilised (e.g. at least partially encapsulated) by a first stabiliser such as a coalescence-resistant surface membrane (for example gelatine), a filmogenic protein (for example an albumin, such as human serum albumin), a polymer material (for example a synthetic biodegradable polymer, an elastic interfacial synthetic polymer membrane, a microparticulate biodegradable polyaldehyde, a microparticulate N-dicarboxylic acid derivative of a polyamino acid-polycyclic imide), a non-polymeric and non-polymerisable wall-forming material, or a surfactant (for example a polyoxyethylene-polyoxypropylene block copolymer surfactant such as a Pluronic, a polymer surfactant, or a film-forming surfactant such as a phospholipid). Preferably, the dispersed gas is in the form of phospholipid-, protein- or polymer-stabilised gas microbubbles. Hence, in one embodiment, the first stabilizer is selected from the group of phospholipids, proteins and polymers. Particularly useful surfactants include phospholipids comprising molecules with net overall negative charge, such as naturally occurring (e.g. soya bean or egg yolk derived), semisynthetic (e.g. partially or fully hydrogenated) and synthetic phosphatidyl-serines, phosphatidylglycerols, phosphatidylinositols, phosphatidic acids and/or cardiolipins. Alternatively, the phospholipids applied for stabilisation may carry an overall neutral charge and be added a negative surfactant such as a fatty acid, e.g. phosphatidylcholine added palmitic acid, or be a mix of differently charged phospholipids, e.g. phosphatidylethanolamines and/or phosphatidylcholine and/or phosphatidic acid and/or phosphatidylserine. For the first stabiliser, i.e. stabilising the microbubble of the first component, different examples are demonstrated in WO2015/047103, Example 5, and Tables 9 and 10, wherein various microbubble formulations with different excipients have been tested. The results demonstrate that the ACT concept used in the current invention is applicable to a wide variety of microbubble formulations, also with regards to the composition of the stabilising membrane. This will also be further demonstrated as suggested in Example 6 of the current disclosure.

The microbubble size of the dispersed gas component intended for intravenous injection should preferably be less than 7 μm, more preferably less than 5 μm and most preferably less than 3 μm in order to facilitate unimpeded passage through the pulmonary system, even when in a microbubble/microdroplet cluster.

### The Second Component (Microdroplet):

The second component comprises a microdroplet comprising an oil phase and a second stabiliser to stabilise said microdroplet, where the oil comprises a diffusible component. This diffusible component is capable of phase shifting upon US insonation of the microbubble/microdroplet cluster and/or diffusing into and/or merging with the gas microbubble of the first component to at least transiently increase the size thereof. For the second component the ‘diffusible component’ is suitably a gas/vapour, volatile liquid, volatile solid or precursor thereof capable of gas generation, e.g. upon administration, the principal requirement being that the component should either have or be capable of generating a sufficient gas or vapour pressure in vivo (e.g. at least 50 torr and preferably greater than 100 torr) so as to be capable of promoting inward diffusion of gas or vapour molecules into the dispersed gas. The ‘diffusible component’ is preferably formulated as an emulsion (i.e. a stabilised suspension) of microdroplets in an appropriate aqueous medium, since in such systems the vapour pressure in the aqueous phase of the diffusible component will be substantially equal to that of pure component material, even in very dilute emulsions.

The diffusible component in such microdroplets is advantageously a liquid at processing and storage temperature, which may for example be as low as −10° C. if the aqueous phase contains appropriate antifreeze material, while being a gas or exhibiting a substantial vapour pressure at body temperature. Appropriate compounds may, for example, be selected from the various lists of emulsifiable low boiling liquids given in the patent applications WO-A-9416379 or WO2015/047103, the contents of which are incorporated herein by reference. Specific examples of emulsifiable diffusible components include aliphatic ethers such as diethyl ether; polycyclic oils or alcohols such as menthol, camphor or eucalyptol; heterocyclic compounds such as furan or dioxane; aliphatic hydrocarbons, which may be saturated or unsaturated and straight chained or branched, e.g. as in n-butane, n-pentane, 2-methylpropane, 2-methylbutane, 2,2-dimethylpropane, 2,2-dimethylbutane, 2,3-dimethylbutane, 1-butene, 2-butene, 2-methylpropene, 1,2-butadiene, 1,3-butadiene, 2-methyl-1-butene, 2-methyl-2-butene, isoprene, 1-pentene, 1,3-pentadiene, 1,4-pentadiene, butenyne, 1-butyne, 2-butyne or 1,3-butadiyne; cycloaliphatic hydrocarbons such as cyclobutane, cyclobutene, methylcyclopropane or cyclopentane; and halogenated low molecular weight hydrocarbons, e.g. containing up to 7 carbon atoms. Representative halogenated hydrocarbons include dichloromethane, methyl bromide, 1,2-dichloroethylene, 1,1-dichloroethane, 1-bromoethylene, 1-chloroethylene, ethyl bromide, ethyl chloride, 1-chloropropene, 3-chloropropene, 1-chloropropane, 2-chloropropane and t-butyl chloride. Advantageously at least some of the halogen atoms are fluorine atoms, for example as in dichlorofluoromethane, trichlorofluoromethane, 1,2-dichloro-1,2-difluoroethane, 1,2-dichloro-1,1,2,2-tetrafluoroethane, 1,1,2-trichloro-1,2,2-trifluoroethane, 2-bromo-2-chloro-1,1,1-trifluoroethane, 2-chloro-1,1,2-trifluoroethyl difluoromethyl ether, 1-chloro-2,2,2-trifluoroethyl difluoromethyl ether, partially fluorinated alkanes (e.g. pentafluoropropanes such as 1H,1H,3H-pentafluoropropane, hexafluorobutanes, nonafluorobutanes such as 2H-nonafluoro-t-butane, and decafluoropentanes such as 2H,3H-decafluoropentane), partially fluorinated alkenes (e.g. heptafluoropentenes such as 1H,1H,2H-heptafluoropent ene, and nonafluorohexenes such as 1H,1H,2H-nonafluorohex-1-ene), fluorinated ethers (e.g. 2,2,3,3,3-pentafluoropropyl methyl ether or 2,2,3,3,3-pentafluoropropyl difluoromethyl ether) and, more preferably, perfluorocarbons. Examples of perfluorocarbons include perfluoroalkanes such as perfluorobutanes, perfluoropentanes, perfluorohexanes (e.g. perfluoro-2-methylpentane), perfluoroheptanes, perfluorooctanes, perfluorononanes and perfluorodecanes; perfluorocycloalkanes such as perfluorocyclobutane, perfluorodimethyl-cyclobutanes, perfluorocyclopentane and perfluoromethylcyclopentane; perfluoroalkenes such as perfluorobutenes (e.g. perfluorobut-2-ene or perfluorobuta-1,3-diene), perfluoropentenes (e.g. perfluoropent-1-ene) and perfluorohexenes (e.g. perfluoro-2-methylpent-2-ene or perfluoro-4-methylpent-2-ene); perfluorocycloalkenes such as perfluorocyclopentene or perfluoro-cyclopentadiene; and perfluorinated alcohols such as perfluoro-t-butanol. Hence, the oil (the diffusible component) of the second component may be selected from the group of aliphatic ethers, heterocyclic compounds, aliphatic hydrocarbons, halogenated low molecular weight hydrocarbons and perfluorocarbons. In one embodiment, the oil phase of the second component comprises a perfluorocarbon.

Particularly useful in the current invention are diffusible components with an aqueous solubility below 1·10⁻⁴ M, more preferably below 1·10⁻⁵ M. It should be noted, however, that if a mixture of diffusible components and/or co-solvents are used, a substantial fraction of the mixture may contain compounds with a higher water solubility. Based on the water solubility, examples of suitable oils (diffusible components) are: perfluorodimethylcyclobutane, perfluoromethylcylopentane, 2-(trifluoromethyl)perfluoropentane and perfluorhexane.

It will be appreciated that mixtures of two or more diffusible components may, if desired, be employed in accordance with the invention; references herein to ‘the diffusible component’ are to be interpreted as including such mixtures.

The second component will also contain material in order to stabilise the microdroplet dispersion, in this text termed ‘second stabiliser’. The second stabiliser may be the same as or different from any materials(s) used to stabilise the gas dispersion, e.g. a surfactant, such as a phospholipid, a polymer or a protein. The nature of any such material may significantly affect factors such as the rate of growth of the dispersed gas phase. In general, a wide range of surfactants may be useful as stabilizers, for example selected from the extensive lists given in EP-A-0727225, the contents of which are incorporated herein by reference. Representative examples of useful surfactants (stabilizers) include fatty acids (e.g. straight chain saturated or unsaturated fatty acids, for example containing 10-20 carbon atoms) and carbohydrate and triglyceride esters thereof, phospholipids (e.g. lecithin), fluorine-containing phospholipids, proteins (e.g. albumins such as human serum albumin), polyethylene glycols, and polymer such as a block copolymer surfactants (e.g. polyoxyethylene-polyoxypropylene block copolymers such as Pluronics, extended polymers such as acyloxyacyl polyethylene glycols, for example polyethyleneglycol methyl ether 16-hexadecanoyloxy-hexadecanoate, e.g. wherein the polyethylene glycol moiety has a molecular weight of 2300, 5000 or 10000), and fluorine-containing surfactants (e.g. as marketed under the trade names Zonyl and Fluorad, or as described in WO-A-9639197, the contents of which are incorporated herein by reference). Particularly useful surfactants include phospholipids comprising molecules with overall neutral charge, e.g. distearoyl-sn-glycerol-phosphocholine (DSPC). For the second component, a range of different stabilisers may be used to stabilise the microdroplet. Further, a wide range of ionic, preferably cationic, substances may be used in order to facilitate a suitable surface charge.

It will be appreciated that, to facilitate attractive electrostatic interactions to achieve clustering between the microbubbles in the first component and the emulsion microdroplets in the second component, these should be of opposite surface charge. Hence, if the microbubbles of the first component are negatively charged, the microdroplets of the second component should be positively charged, or vice versa. In a preferred embodiment, the surface charge of the microbubbles of the first component is negative, and the surface charge of the microdroplets of the second component is positive. In order to facilitate a suitable surface charge for the oil microdroplets a cationic surfactant may be added to the stabilising structure. A wide range of cationic substances may be used, for example at least somewhat hydrophobic and/or substantially water-insoluble compounds having a basic nitrogen atom, e.g. primary, secondary or tertiary amines and alkaloids. A particularly useful cationic surfactant is stearylamine. In one embodiment, the second stabiliser is a neutral phospholipid added a cationic surfactant, for example such as DSPC-membrane with stearylamine.

In one embodiment, the first stabiliser and the second stabiliser each independently comprises a phospholipid, a protein, a polymer, a polyethyleneglycol, a fatty acid, a positively charged surfactant, a negatively charged surfactant or mixtures thereof.

In one embodiment, the first component comprises a dispersed gas selected from the group of sulphur hexafluoride, perfluoropropane, perfluorobutane, perfluoropentane, perflurohexane, nitrogen and air or a mix thereof, stabilised by a first stabiliser selected from the group of phospholipids, proteins and polymers; the second component comprises a diffusible component selected from the group of perfluorocarbons, e.g. a perfluorocycloalkane, stabilised with a second stabiliser selected from the group of surfactants, polymers and proteins. More particularly, the first stabilizer comprises a phospholipid, a protein, or a polymer optionally added a negatively charged surfactant, and the second stabilizer comprises a phospholipid, protein, or a polymer optionally added a positively charged surfactant.

In one embodiment, the first component comprises a dispersed gas selected from the group of sulphur hexafluoride, perfluoropropane, perfluorobutane, perfluoropentane, perflurohexane, or a mix thereof, stabilized by a first stabilizer selected from the group of phospholipids, proteins and polymers; the second component comprises a diffusible component selected from the group of perfluorocarbons, e.g. a perfluorocycloalkane, stabilized with a second stabilizer selected from the group of surfactants, e.g. including phospholipids, polymers and proteins. More specifically, either of the stabilizers are selected from phospholipids.

In some embodiments, the cluster composition comprises a suspension of clusters in an aqueous biocompatible medium, where said clusters have a mean diameter in the range 1 to 10 μm, and a circularity <0.9 and comprises:

(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions;
where the first and second stabilisers are both selected from the list comprising, but not limited to coalescence-resistant surface membranes, filmogenic proteins, polymer materials, non-polymeric and non-polymerisable wall-forming materials, surfactants, and are preferably selected from proteins, polymers and phospholipids, and are identical or different.

In some embodiments, the cluster composition comprises a suspension of clusters in an aqueous biocompatible medium, where said clusters have a mean diameter in the range 1 to 10 μm, and a circularity <0.9 and comprises:

(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions;
where the first and second stabilisers are both surfactants.

In some embodiments, the cluster composition comprises a suspension of clusters in an aqueous biocompatible medium, where said clusters have a mean diameter in the range 1 to 10 μm, and a circularity <0.9 and comprises:

(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions;
where the first and second stabilisers are both selected from the list comprising, but not limited to coalescence-resistant surface membranes, filmogenic proteins, polymer materials, non-polymeric and non-polymerisable wall-forming materials, surfactants, and are identical or different;
where the diffusible component is an emulsifiable low boiling liquid with an aqueous solubility below 1·10⁻⁴ M, such as below 1·10⁻⁵ M.

In some embodiments, the cluster composition comprises a suspension of clusters in an aqueous biocompatible medium, where said clusters have a mean diameter in the range 1 to 10 μm, and a circularity <0.9 and comprises:

(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions;
where the first and second stabilisers are both surfactants.

where the diffusible component is an emulsifiable low boiling liquid with an aqueous solubility below 1·10⁻⁴ M, such as below 1·10⁻⁵ M.

It is envisioned that the ACT concept for delivery of antimicrobial agent or treatment of an infection, i.e. the composition for use and methods of the invention, is a concept that applies for a broad combination of components (first and second) components, and also for a wide range of antimicrobial agents. Hence, any of the ingredients listed for the first component, including gases and first stabilizers, can be combined with the ingredients listed for the second component, including the diffusible component and the second stabilizers. To summarize, in one embodiment, the two-component formulation system for preparation of the microbubble/microdroplet cluster composition, for use according to the methods of the invention, comprises:

(i) a first component which comprises a gas microbubble and a first stabilizer to stabilize said microbubble, wherein the gas of the gas microbubble is selected from the group of halogenated gases, preferably is a perfluorinated gas, and most preferably is perfluorobutane; and the first stabilizer is selected from the group of phospholipids, proteins and polymers, optionally added a negatively charged surfactant, and more preferably is a phospholipid, and most preferably is hydrogenated egg phosphatidyl serine-sodium (HEPS-Na); and
(ii) a second component which comprises a microdroplet comprising an oil phase and a second stabilizer to stabilize said microdroplet, where the oil comprises a diffusible component capable of diffusing into said gas microbubble so as to at least transiently increase the size thereof, wherein the oil is selected from the group of aliphatic ethers, heterocyclic compounds, aliphatic hydrocarbons, halogenated low molecular weight hydrocarbons and perfluorocarbons, is preferably a perfluorocarbon, and is most preferably perfluoromethyl-cyclopentane (pFMCP); and the second stabilizer is selected from the group of phospholipids, polymers and proteins, optionally added a positively charged surfactant, is more preferably a phospholipid added a positively charged surfactant, and is most preferably 1,2-distearoyl-sn-glycerol-3-phosphocholine (DSPC) added stearylamine (SA);
where the microbubbles and microdroplets of the first and second components have opposite surface charges and form said clusters via attractive electrostatic interactions.

The first and second components of the two-component formulation system are combined shortly before the intended use, for preparation of a composition of microbubble/microdroplet clusters, and for use in an appropriate time window according to the methods of the invention. Hence, in one embodiment of the invention, the method comprises a step of preparing the microbubble/microdroplet cluster composition prior to the administration step. In a preferred embodiment, the microbubble/microdroplet cluster composition is prepared by reconstitution of the first component (microbubbles) in dry powder form with the second component (microdroplets) in fluid form. It will also be appreciated that the mixing of the first and second components can be achieved in various manners known to the skilled person, depending on the form of the components; e.g. mixing two fluid components, reconstitution of one component in dry powder form with one component in fluid form, mixing two components in dry form prior to reconstitution with fluid (e.g. water for injection or buffer solution). Also, it will be appreciated that other components may influence the ability of the microbubbles and microdroplets to form clusters upon mixing including, but not limited to; the level of surface charge of the microbubbles/microdroplets, the concentration of the microbubbles/microdroplets in the two components, the size of the microbubbles/microdroplets, the composition and concentration of ions, the composition and concentration of excipients (e.g. buffer or tonicity components) etc. (see WO2015/047103, Example 1). Such characteristics of the components and the composition may also influence the size and stability (both in vitro and in vivo) of the clusters generated and may be important factors influencing biological attributes (e.g. efficacy and safety profile). It is also appreciated that not all of the microbubbles/microdroplets in the cluster composition may be present in clustered form, but that a substantial fraction of the microbubbles and/or microdroplets may be present together in a free (non-clustered) form together with a population of microbubble/microdroplet clusters. In addition, the way the two components are mixed may influence these aspects, including, but not limited to; shear stress applied during homogenisation (e.g. soft manual homogenisation or strong mechanical homogenisation) and time range for homogenisation.

The microdroplet size of the dispersed diffusible component in emulsions intended for intravenous injection should preferably be less than 7 μm, more preferably less than 5 μm, most preferably less than 4 μm, and greater than 0.5 μm, more preferably greater than 1 μm, most preferably greater than 2 μm in order to facilitate unimpeded passage through the pulmonary system, but still retain a volume that is sufficient for activated bubble retention in the microvasculature. In a preferred embodiment, the mean diameter of the microdroplets in the second component is between 2 to 4 μm. Growth of the dispersed gas phase in vivo may, for example, be accompanied by expansion of any encapsulating material (wherein this material has sufficient flexibility) and/or by abstraction of excess surfactant from the administered material to the growing gas-liquid interfaces. It is also possible, however, that stretching of the encapsulating material and/or interaction of the material with ultrasound may substantially increase its porosity. Whereas such disruption of encapsulating material has hitherto in many cases been found to lead to rapid loss of echogenicity through outward diffusion and dissolution of the gas thereby exposed, the inventors have found that when using compositions in accordance with the present invention, the exposed gas exhibits substantial stability. Whilst not wishing to be bound by theoretical calculations, the inventors suggest that the exposed gas, e.g. in the form of liberated microbubbles, may be stabilised, e.g. against collapse of the microbubbles, by a supersaturated environment generated by the diffusible component, which provides an inward pressure gradient to counteract the outward diffusive tendency of the microbubble gas. The exposed gas surface, by virtue of the substantial absence of encapsulating material, may cause the activated bubbles to exhibit exceptionally favourable acoustic properties as evidenced by high backscatter and low energy absorption (e.g. as expressed by high backscatter: attenuation ratios) at typical diagnostic imaging frequencies; this echogenic effect may continue for a significant period, even during continuing ultrasound irradiation.

### The Ultrasound Procedures—Activation and Enhancement Steps

It will be appreciated that the microbubble of the first component and the large bubbles formed after activation of the clusters are different in size and hence will respond differently to a given US field. Hence, the US fields applied in steps iii) (the Activation step) and iv) (the Enhancement step), when using the cluster composition in a method of treatment or delivery, need to be different and carefully selected in order to facilitate increased extravasation of the co-administered antimicrobial agent.

### The Activation Step

The microbubbles of the first component are typically 2-3 μm in diameter and their acoustic resonance is within the diagnostic frequency range (1-10 MHz). When the cluster composition has been administered to the subject, activation of the clusters is readily obtained with standard diagnostic ultrasound imaging pulses used for example in conventional medical ultrasound abdominal and cardiac applications, at mid-range to low mechanical indices, i.e. an MI below 1.9, such as below 0.7, preferably below 0.4 such as below 0.3, but above 0.1. Upon such insonation (activation insonation), the oil in the microdroplet vaporises, forming a large (10 μm or more in diameter). The median diameter of microcapillaries in the human body is approx. 7 μm in diameter, hence, due to their size the activated bubbles transiently trap in the microvasculature of the region of interest (i.e. site of infection). After activation, the gas within the large bubbles starts to diffuse into the blood stream and they shrink slowly until they are small enough to dislodge and become free-flowing after approx. 5-10 minutes. Activation under medical ultrasound imaging control allows spatially targeted activation of the clusters in the tissue region being insonated by the ultrasound field, i.e. the region of interest, i.e. the site of infection. Hence, the method allows for spatially targeted extravasation of the co-administered antimicrobial agent, specifically to the site of infection.

In a preferred embodiment, the US field applied during the activation step has a first frequency in the range of 1-10 MHz and an MI of less than 1.9, preferably less than 0.7 and most preferably less than 0.4.

In one embodiment, the activation, i.e. the US insonation at the first frequency, starts immediately after each administration of the cluster composition, such as within 20 seconds, and lasts for e.g. 60-120 seconds. In one embodiment, when imaging is performed, this is performed before and during injection of the cluster composition, and then the activation clock is started when inflow of contrast is seen.

### Optional Imaging Step

The clusters are not activated at low MI (below the cluster activation threshold of approx. 0.1) allowing standard medical ultrasound contrast agent imaging to be performed, for example to identify relevant pathology without activation of the clusters. Hence, in one embodiment the method includes a step of imaging using low MI contrast agent imaging modes (MI<0.15, such as MI<0.1) to image the microbubble component, i.e. the dispersed gas, without activation of the clusters, to identify the pathology region (site of infection) for treatment. Hence, as the clusters are not activated at low MI (below the activation threshold) standard medical ultrasound contrast agent imaging may be performed, prior to the activation step, for example to identify microvascular pathology, e.g. targeted tissue.

### The Enhancement Step

After activation, due to being larger than the microcapillaries, the large phase shift bubbles produced are transiently trapped in the microvasculature of the insonated region of interest, i.e. the site of infection. The resulting large phase shift bubbles are approximately 1000 times the volume of the emulsion microdroplet vaporised (a 20 μm bubble diameter formed from a 2 μm diameter oil microdroplet). The scattering cross sections of these large phase shift bubbles are orders of magnitude greater than the scattering cross sections of the micron sized microbubbles comprised in the clusters before activation. As a result, the large phase shift bubbles produce copious backscatter signal and are readily imaged in fundamental imaging mode with diagnostic imaging systems. The mechanical resonance frequencies of the large phase shift bubbles are also an order of magnitude lower than the resonance frequencies of the microbubbles comprised in the clusters before activation; around 0.5 MHz vs. 3-5 MHz. Application of acoustic fields commensurate with the resonance frequencies of the larger phase shift bubbles produces relatively large radius oscillations at MI's within the medical diagnostic range. The applicant has investigated attributes of the US fields applied during the second insonation step (the Enhancement step) and their effect on the functionality of the applied procedure. Surprisingly, and contrary to the teachings of WO2015/047103, where the preferred frequency range is disclosed to between 0.2 to 1 MHz and an MI of <0.4 is suggested, the applicant has found that the functionality of the concept is quite sensitive to these parameters. Based on these studies, the applicant has found that a preferred frequency range is between 0.4 to 0.6 MHz and that the MI applied should be kept to more than 0.1, but less than 0.3. With lower frequencies and higher MI than this during the Enhancement step, step (iv), the applicant has surprisingly found that the activated bubble oscillations induced are too strong, leading to a significant loss of efficacy and vascular damage. On the other hand, with higher frequencies and lower MIs, the bubble oscillations induced are too small, leading to a lack of sufficient biomechanical effects and hence a significant loss in therapeutic efficacy. Thus, low frequency ultrasound, in the range of 0.05 to 2 MHz, such as 0.1 to 1.5 MHz or such as 0.2 to 1 MHz may be used, however as a preferred embodiment a frequency of 0.4 to 0.6 MHz, combined with an MI between 0.1 to 0.3 is rather applied to produce the bio-effect mechanisms that enhance the uptake of the administered drug, and hence facilitates extravasation. In one embodiment, the frequency of the insonation in the enhancement step is lower than the frequency of the insonation of the activation step. Exploiting the resonance effects of the activated bubbles allows better control of initiation of these bio-effects at lower acoustic intensities and at lower frequencies than possible with other technologies. Coupled with the fact that the large phase shift bubbles are activated and deposited in the tissue microvasculature under imaging control (allow spatial targeting of the large activated bubbles in tissue), and their prolonged residence time, allows more efficient and controlled implementation of the drug delivery mechanisms.

Further application of ultrasound after activation hence facilitates extravasation by effectively open biological barriers and increase the therapeutic effect of the antimicrobial drug delivered to the site of infection.

The insonation with low frequency ultrasound follows the activation step and should typically last for 3 to 10 minutes, such as for about 5 minutes. There is preferably an immediate start of step (iv) after step (iii). A dual frequency transducer may beneficially be used in the treatment, for both the activation step and the enhancement step. By using such, the switch from the activation insonation in step (iii) to the enhancement insonation in step (iv) can be made without any delay. Application of the enhancement field immediately after activation may be important for the resulting therapeutic benefit. In this respect it would be beneficial to apply both the activation and the enhancement insonation using a broad band or dual frequency US transducer. I.e. a transducer capable of delivering sufficient US pressure (i.e. MI) over all frequencies required by the stated preferred ranges. E.g. a transducer capable of delivering MIs of up to 0.4 at both 1 to 10 MHz and at 0.1 to 1 MHz, more preferably 0.4 to 0.6 MHz.

It is envisioned that the dual action concept for drug delivery to the site of infection and treatment of the infection, i.e. the composition for use of the invention, is a concept that applies for a broad combination of first and second components, and also for a range of infectious diseases and antimicrobial agents.

In the system or method of the invention, or in the pharmaceutical composition for use, an antimicrobial agent may be loaded into the microdroplets of the second component for release at targeted site in vivo upon activation. Example 6 of WO 2015/047103 A1 provides results from a fluorescence microscopy study on activated bubbles made loaded with Nile Red fluorescence dye. It is demonstrated that, after activation, the loaded substance is homogeneously expressed at the surface of the activated bubbles and will hence be in close contact with the endothelial wall and accessible for extravasation. Examples 8 and 5-4 of the same elucidates concepts to achieve such loading. Both examples are incorporated herein by reference.

### Infectious Diseases and Therapeutic Agents for Treatment

The infection to be treated using a composition or method according to the invention may be subclinical, or silent, without a clinically apparent infection, or it may be clinical and apparent. The infection may be latent. The infection may result from a primary pathogen or an opportunistic pathogen. In some embodiments, the infection is a primary infection. In other embodiments, the infection is a secondary infection. The infection may be a mixed, iatrogenic, nosocomial, and/or community-acquired infection.

In some embodiments, the infection results from an invasive medical procedure, such as on the site of a surgical incision, catheter, IV, hypodermic, blood sample and/or biopsy.

In some embodiments, the infection is selected from the group comprising, but not limited to, general cellulitis, ear infections, eye infections, sinusitis, food poisoning, skin infections, furuncles, folliculitis, scalded skin syndrome, general wound infections, necrotizing fascitiitis, lung infections, pneumonia, toxic shock syndrome, actinomycosis, nocardiosis, meningitis, and sepsis.

Certain vaccines need to be given several times to make a strong immune response. In some embodiments, the ACT treatment according to the invention can enhance this immune response and reduce the necessary number of vaccinations.

In some embodiments, the infection to be treated using a composition or method according to the invention is a bacterial infection. The infection may be caused by one or more types of Gram-positive and/or Gram-negative bacteria. In certain embodiments, the infection is caused by one or more types of bacteria from the list comprising, but not limited to, Staphylococcus, Staphylococcus aureus, Hemophilus, Hemophilus influenzae, Pseudomonas, Pseudomonas aeruginosa, Streptococcus, Streptococcus pneumoniae, Streptococcus Group A, Group B, Group C, Group D, Group G, Mycobacterium, Mycobacterium tuberculosis, Clostridium, and Enterobacteriaceae. In specific embodiments, the infection is caused by antibiotic drug resistant strain of bacteria.

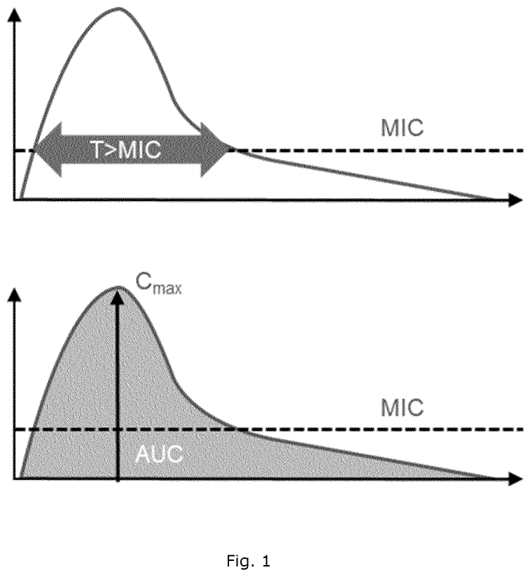

The pharmacology of antimicrobial therapy can be divided into two distinct components. The first of these components is pharmacokinetics (PK), which examines how the body handles drugs, including absorption, distribution, metabolism and elimination, and the second component is pharmacodynamics (PD), which examine the relationship between drug PK, a measure of in vitro potency (usually the minimum inhibitory concentration [MIC]), and the treatment outcome (usually efficacy or sometimes drug toxicity). The time course of antimicrobial activity is a reflection of the interrelationship between PK and PD. PK/PD relationships are vital in facilitating the translation of microbiological activity into clinical situations and ensuring that antimicrobial agents achieve a successful outcome. A large number of studies have indicated that antimicrobial agents can be divided into two major classes: those that exhibit concentration-dependent killing and prolonged persistent effects (e.g. aminoglycosides, fluoroquinolones), for which the peak plasma concentration in relation to the Minimum Inhibitory Concentration (MIC) of the organism causing the infections (Cmax/MIC) is the major PK/PD parameter which correlates best with efficacy; the other class is those antimicrobial agents that exhibit time-dependent killing and minimal-to-moderate persistent effects (e.g. beta-lactam and macrolide classes), the time (expressed as a percentage of the dosing interval) that drug concentration exceeds the MIC (% T>MIC) is the major parameter determining efficacy. The difference of these two classes of antimicrobial agents is illustrated in FIG. 1.

In Example 2, it is clearly demonstrated that application of the ACT procedure according to the current invention, significantly increases the concentration of co-administered agents in the targeted tissue compartment. In Study 1 of Example 2, a 100% increase in the uptake of a drug mimicking molecule, specifically to the tissue targeted by the US insonation, is demonstrated. Furthermore, in Study 2 of Example 2, ACT was demonstrated to clearly increase the permeability of the Blood Brain Barrier (BBB) for a large, nanoparticulate drug construct when applying the two-step insonation approach of the current invention. The blood-brain-barrier (BBB) represents the tightest vascular barrier in the body. Unimpaired, it completely closed to therapeutic agents larger than approximately 4-500 Daltons, prevailing medicinal treatment of most diseases and disorders of the central nervous system (CNS). ACT resulted in a more than 290-470% increase in the extravasation to the brain parenchyma, localised to the insonated region of interest. Hence, ACT enables delivery of small and large drug constructs, even across the tightest vascular barrier in the body (the BBB). These two studies hence demonstrate the ability of ACT to locally increase the concentration of co-administered therapeutic agents in a targeted tissue compartment and indirectly, beyond reasonable doubt, that the concept will enhance the therapeutic benefit of antimicrobial agents. Based on the PK/PD characteristics, the combination of ACT with Cmax-dependent agents could be particularly beneficial—the primary attribute of the ACT procedure is to increase the tissue concentration of the co-administered drug. However, as ACT also may improve distribution of drug in the targeted tissue, i.e. enhance the penetration of the drug and its chance to reach the pathological microbes, the % T>MIC class of agents may also benefit from ACT. Furthermore, for % T>MIC agents, in certain cases it is important to reach higher concentrations when MIC is increased because of resistance; for example, multi-drug-resistant (MDR) gram negative bacterial infections, such as of the prostate. In this case, even though prolonged infusion is usually the dosing adaptations, the treatment regimen would typically be started with a “loading dose” [N. J. Rhodes et al., Impact of Loading Doses on the Time to Adequate Predicted Beta-Lactam Concentrations in Prolonged and Continuous Infusion Dosing Schemes, Clinical Infectious Diseases, Volume 59, Issue 6, 15 September 2014, Pages 905-907], to rapidly achieve a higher than MIC plasma concentration in the infected tissue. In this case, i.e. in combination with a loading dose, ACT could represent a very clinically valuable intervention. For example, for a meropenem loading dose, the infusion would be over maximum 30 minutes—imminently suitable for a concurrent ACT procedure. Lastly, after an ACT procedure, the increased penetrability of the vascular barrier can last for some time, i.e. more than 1 hour or more than 2-3 hours. In this case, the time the tissue concentration is above MIC may be substantially prolonged and the therapeutic efficacy of T %>MIC agents significantly improved.

Hence, as the therapeutic efficacy of all antimicrobial agents to some extent are dose/concentration dependent, vs. drug alone, it is highly likely that a combination with ACT will lead to an improved therapeutic outcome with both classes of agents during treatment of any focal/localized types of infection. The therapeutic benefit of combining ACT with Levofloxacin treatment of Staphylococcus infection in mice is clearly indicated by Example 3 and will be demonstrated by the prospective Example 4 and Example 5.

Antimicrobial agents and classes of agents that are useful under the current invention include, but are not limited to; anti-infectives; amebicides; aminoglycosides; anthelmintics; antiparasitics such as antiprotozoals, ectoparasiticides, antifungals such as azole antifungals, echinocandins, polyenes; antimalarial agents such as antimalarial combinations, antimalarial quinolines; antituberculosis agents such as amino salicylates, antituberculosis combinations, diarylquinolines, hydrazide derivatives, nicotinic acid derivatives, rifamycin derivatives, Streptomyces derivatives; antiviral agents such as adamantane antivirals, antiviral boosters, antiviral combinations, antiviral interferons, chemokine receptor antagonists, integrase strand transfer inhibitors, neuraminidase inhibitors, non-nucleoside reverse transcriptase inhibitors (NNRTIs), nonstructural protein 5A (NS5A) inhibitors, nucleoside reverse transcriptase inhibitors (NRTIs), protease inhibitors, purine nucleosides; carbapenems; carbapenems/beta-lactamase inhibitors; cephalosporins such as cephalosporins/beta-lactamase inhibitors, first generation cephalosporins, fourth generation cephalosporins, next generation cephalosporins, second generation cephalosporins, third generation cephalosporins; glycopeptide antibiotics; glycylcyclines; leprostatics; lincomycin derivatives; macrolide derivatives such as ketolides, macrolides; miscellaneous antibiotics; oxazolidinone antibiotics; penicillins such as aminopenicillins, antipseudomonal penicillins, beta-lactamase inhibitors, natural penicillins, penicillinase resistant penicillin's; quinolones; streptogramins; sulphonamides; tetracyclines; urinary anti-infectives; new classes of agents in development, such as immunotherapeutic agents, such as checkpoint inhibitors.

In a preferred embodiment, the antimicrobial agent to be used in conjunction with ACT, i.e. in the method of the invention, is selected from the Cmax dependent class for drugs.

In another preferred embodiment, the antimicrobial agent to be used in conjunction with ACT is selected from the % T>MIC dependent class of drugs.

In one embodiment the antimicrobial agent is an antibiotic. In another embodiment the antimicrobial agent is an antifungal agent. In yet another embodiment, the antimicrobial agent is an antiviral, and in yet another embodiment the microbial agent is an antiparasitic.

The antimicrobial agents used in the treatment of bacterial infections are antibiotics. The antibiotics that may be used in the composition or the method according to the invention may have any mechanism of action known to the skilled person. Non-limiting examples include: agents acting on the bacterial cell wall such as bacitracin, the cephalosporins, cycloserine, fosfomycin, the penicillins, ristocetin, and vancomycin; agents affecting the cell membrane or exerting a deterging effect, such as colistin, novobiocin and polymyxins; agents affecting cellular mechanisms of replication, information transfer, and protein synthesis by their effects on ribosomes, e.g., the aminoglycosides, the tetracyclines, chloramphenicol, clindamycin, cycloheximide, fucidin, lincomycin, puromycin, rifampicin, other streptomycins, and the macrolide antibiotics such as erythromycin and oleandomycin; agents affecting nucleic acid metabolism, e.g., the fluoroquinolones, actinomycin, ethambutol, 5-fluorocytosine, griseofulvin, rifamycins; and drugs affecting intermediary metabolism, such as the sulfonamides, trimethoprim, and the tuberculostatic agents isoniazid and para-aminosalicylic acid. The antibiotics may be broad-spectrum or “narrow-spectrum”. They may have one or more primary mechanism of action. They may be used separately or in combination with other antimicrobial agents, such as in combination with other antibiotics.

In some embodiments, the antimicrobial agent is an antibiotic chosen from the list comprising amoxicillin, ceftriaxon, doxycycline, cephalexin, ciprofloxacin, clindamycin, metronidazole, azithromycin, levofloxacin, sulfamethoxazole and trimethoprim, amoxicillin and clavulanate, levofloxacin.

In some embodiments, the antibiotic is chosen from the list comprising amoxicillin/clavulanate, Amoxil, Augmentin, azithromycin, Azithromycin Dose Pack, Bactrim, Bactrim DS, ceftriaxone, cefuroxime, Cipro, Cleocin, Flagyl, Keflex, Levaquin, levofloxacin, Penicillin VK, sulfamethoxazole/trimethoprim, vancomycin, Zithromax, Rocephin, Avelox, Ceftin, minocycline, Vibramycin, Doxy 100, moxifloxacin, penicillin v potassium, Septra, Zyvox, Apo-Amoxi, cilastatin/imipenem, cefazolin, Doryx, Doryx MPC, gentamicin, Monodox, Morgidox, Oraxyl, Septra DS, Cipro I.V., Cipro XR, Cleocin HCl, Flagyl 375, Flagyl IV, linezolid, tobramycin, Amoclan, ampicillin, Augmentin XR, chloramphenicol, Cleocin Pediatric, Cleocin Phosphate, Co-trimoxazole, Minocin, tetracycline, Vancocin, Vancocin HCl, Zinacef, Achromycin V, Actisite, Ala-Tet, Azactam, Bicillin L-A, Brodspec, Chloromycetin, Dynacin, Garamycin, Lincocin, Minocin for Injection, Sulfatrim Pediatric, Tobi, Unasyn, Vancocin HCl Pulvules, Ximino, ampicillin/sulbactam, Avelox I.V., aztreonam, Bactocill, Cefotan, cefotetan, cefoxitin, Chloromycetin Sodium Succinate, Declomycin, demeclocycline, lincomycin, nafcillin, oxacillin, penicillin g benzathine, penicillin g potassium, Penicillin G Procaine, penicillin g sodium, Pfizerpen, Primaxin IV, procaine penicillin, Sivextro, tedizolid, TOBI Podhaler.

In some embodiments, the antibiotic is chosen from the list comprising amoxicillin/clavulanate, Amoxil, Augmentin, azithromycin, Bactrim, Flagyl, Keflex, Levaquin, levofloxacin, Penicillin VK, sulfamethoxazole/trimethoprim, vancomycin, Zithromax, Rocephin, Avelox, Ceftin, minocycline, Vibramycin, moxifloxacin, penicillin v potassium, Septra, Zyvox, Apo-Amoxi, cilastatin/imipenem, cefazolin, Doryx, gentamicin, Monodox, Morgidox, Oraxyl, Septra DS, Cipro I.V., Cipro XR, Cleocin HCl, Flagyl 375, linezolid, tobramycin, Amoclan, ampicillin, chloramphenicol, Cleocin Phosphate, Co-trimoxazole, Minocin, tetracycline, Vancocin, Zinacef, Achromycin V, Actisite, Dynacin, Garamycin, Lincocin, Unasyn, Vancocin HCl Pulvules, Ximino, ampicillin/sulbactam, Avelox I.V., aztreonam, Bactocill, Cefotan, cefotetan, cefoxitin, lincomycin, nafcillin, oxacillin, penicillin g benzathine, penicillin g potassium, Penicillin G Procaine, penicillin g sodium, procaine penicillin, Sivextro, tedizolid.

In some embodiments, the antibiotic is chosen from the list comprising Augmentin, Azitromycin, Cefuroksim, Flagyl, Flagyl ER, Amoxil, Cipro, Keflex, Bactrim, Bactrim DS, Levaquin, Zithromax, Avelox, Cleocin, Vancomycin, Rocephin.

In some embodiments, the infection to be treated using a composition or method according to the invention is a fungal infection. The infection may be an infection from one or more of Ascomycota, including yeasts such as Candida, filamentous fungi such as Aspergillus, Pneumocystis species, and dermatophytes, a group of organisms causing infection of skin and other superficial structures in humans, and Basidiomycota, including the human-pathogenic genus Cryptococcus.

Whereas bacteria and fungi are different and phylogenetically distant groups, the list of similarities is also considerable:
Both fungi and bacteria have cell walls.
Most bacteria and all fungi obtain energy from aerobic respiration.
Both groups possess cell membranes composed of phospholipids
Some yeasts and most bacteria reproduce by binary fission.
Certain bacteria and certain fungi have the ability to produce antibiotics.
Many species of both groups are human animal and plant pathogens.
Both groups have the ability to survive harsh environmental conditions by producing specialised thick-walled spores, although with different formation and structures.

The antimicrobial agents used in the treatment of fungal infections are antifungals. The antifungals that may be used in the composition or the method according to the invention may have any mechanism of action known to the skilled person. They may be used separately or in combination with other antimicrobial agents.

In some embodiments, the antifungal is chosen from the list comprising, but not limited to, polyenes, azoles such as imidazoles, triazoles and thiazoles, allylamines, echinocandins.

In some embodiments, the antifungal is chosen from the list comprising clotrimazole, econazole, miconazole, terbinafine, fluconazole, ketoconazole, amphotericin, gallium nitrate.

In one embodiment, the antifungal is the drug Ganite, gallium nitrate. Although gallium has no natural function in biology, gallium ions interact with cellular processes in a manner similar to iron(III). When gallium ions are mistakenly taken up in place of iron(III) by bacteria such as Pseudomonas, the ions interfere with respiration, and the bacteria die. This happens because iron is redox-active, allowing the transfer of electrons during respiration, while gallium is redox-inactive.

In some embodiments, the infection to be treated using a composition or method according to the invention is a viral infection. The antimicrobial agents used in the treatment of viral infections are antivirals. The antivirals that may be used in the composition or the method according to the invention may have any mechanism of action known to the skilled person. They may be used separately or in combination with other antimicrobial agents.

In some embodiments, the antiviral is chosen from the list comprising Idoxuridine, Trifluridine, Brivudine, Vidarabine, Entecavir, Telbivudine, Foscarnet, Zidovudine, Didanosine, Zalcitabine, Stavudine, Lamivudine, Lamivudine+zidovudine, Abacavir, Abacavir+lamivudine+zidovudine, Emtricitabine, Nevirapine, Delavirdine, Efavirenz, Etravirine, Rilpivirine, Saquinavir, Ritonavir, Indinavir, Nelfinavir, Amprenavir, Lopinavir-ritonavir, Atazanavir, Fosamprenavir, Tipranavir, Darunavir, Darunavir+cobicistat, Atazanavir+cobicistat, Telaprevir, Boceprevir, Simeprevir, Asunaprevir, Vaniprevir+ribavirin+PegIFNa-2b, Paritaprevir, Grazoprevir, Raltegravir, Elvitegravir, Dolutegravir, Dolutegravir+abacavir+lamivudine, Dolutegravir+lamivudine, RSV-IGIV, Palivizumab, Docosanol, Enfuvirtide, Maraviroc, VZIG, VariZIG, Acyclovir, Ganciclovir, Famciclovir, Valacyclovir, Penciclovir, Valganciclovir, Cidofovir, Tenofovir disoproxil fumarate, Adefovir dipivoxil, Tenofovir disoproxil fumarate+emtricitabine, Tenofovir disoproxil fumarate+efavirenz+emtricitabine, Tenofovir disoproxil fumarate+rilpivirine+emtricitabine, Tenofovir disoproxil fumarate+cobicistat+emtricitabine+elvitegravir, Tenofovir alafenamide+cobicistat+emtricitabine+elvitegravir, Tenofovir alafenamide+rilpivirine+emtricitabine, Tenofovir alafenamide+emtricitabine, Sofosbuvir+ribavirin, Sofosbuvir+ribavirin+PegIFNa, Daclatasvir+asunaprevir, Ledipasvir+sofosbuvir, Sofosbuvir+simeprevir, Ombitasvir+dasabuvir+paritaprevir+ritonavir, Ombitasvir+paritaprevir+ritonavir, Daclatasvir+sofosbuvir, Elbasvir+grazoprevir, Amantadine, Ribavirin, Rimantadine, Zanamivir, Oseltamivir, Laninamivir octanoate, Peramivir, Favipiravir, Pegylated interferon alfa 2b, Interferon alfacon 1, Pegylated interferon alfa 2b+ribavirin, Pegylated interferon alfa 2a, Fomivirsen, Podofilox, Imiquimod, Sinecatechins.

In some embodiments, the antiviral is chosen from the list comprising Idoxuridine, Trifluridine, Brivudine, Vidarabine, Entecavir, Telbivudine, Foscarnet, Zidovudine, Didanosine, Zalcitabine, Stavudine, Lamivudine, Abacavir, Emtricitabine, Nevirapine, Delavirdine, Efavirenz, Etravirine, Rilpivirine, Saquinavir, Ritonavir, Indinavir, Nelfinavir, Amprenavir, Lopinavir-ritonavir, Atazanavir, Fosamprenavir, Tipranavir, Darunavir, Telaprevir, Boceprevir, Simeprevir, Asunaprevir, Paritaprevir, Grazoprevir, Raltegravir, Elvitegravir, Dolutegravir, RSV-IGIV, Palivizumab, Docosanol, Enfuvirtide, Maraviroc, VZIG, VariZIG, Acyclovir, Ganciclovir, Famciclovir, Valacyclovir, Penciclovir, Valganciclovir, Cidofovir, Tenofovir disoproxil fumarate, Adefovir dipivoxil, Amantadine, Ribavirin, Rimantadine, Zanamivir, Oseltamivir, Laninamivir octanoate, Peramivir, Favipiravir, Pegylated interferon alfa 2b, Interferon alfacon 1, Pegylated interferon alfa 2a, Fomivirsen, Podofilox, Imiquimod, Sinecatechins.

In some embodiments, the antiviral is chosen from the list comprising Idoxuridine, Trifluridine, Brivudine, Didanosine, Zalcitabine, Emtricitabine, Nevirapine, Delavirdine, Efavirenz, Etravirine, Rilpivirine, Saquinavir, Ritonavir, Indinavir, Nelfinavir, Amprenavir, Lopinavir-ritonavir, Atazanavir, Fosamprenavir, Tipranavir, Darunavir, Telaprevir, Boceprevir, Simeprevir, Asunaprevir, Paritaprevir, Grazoprevir, RSV-IGIV, Palivizumab, Rimantadine, Zanamivir, Oseltamivir, Laninamivir octanoate, Peramivir, Favipiravir.

In some embodiments, the infection to be treated using a composition or method according to the invention is a parasitic infection. The parasites causing the infection may be unicellular microrganisms (protozoa) and/or multicellular organisms with organ systems (helminths). The antimicrobial agents used in the treatment of parasitic infections are antiparasitics. The antiparasitics that may be used in the composition or the method according to the invention may have any mechanism of action known to the skilled person. They may be used separately or in combination with other antimicrobial agents.

In some embodiments, the infection to be treated using a composition or method according to the invention is a parasitic infection.

There is a need for treatment against parasites that can cross the blood-brain barrier (BBB) and enter the central nervous system (CNS). Common antiparasitics do not cross the BBB and do therefore not work if the parasites cross the BBB. Hence there is a great need to facilitate the crossing of the BBB by antiparasitics. As demonstrated by Example 5, ACT is capable of delivering even large drug molecules and constructs across the BBB and into the brain parenchyma. Hence, in a preferred embodiment, the composition and the method of the current invention is for treatment of infectious diseases of the CNS.

Taenia solium, also known as the pork tapeworm, can cause epileptic seizures and other neurological problems in humans, from the ingestion of eggs containing infective larvae. The breakdown of the egg shell occurs in the intestines, allowing the larvae to exit and enter the bloodstream. Once in the circulation, larvae may settle in many types of body tissues. Larvae may cross the BBB and enter the CNS, where the embryos develop into fluid-filled cysts leading to a condition known as neurocysticercosis, which is one of the most dangerous parasitic CNS infections worldwide. Diagnosis of neurocysticercosis is difficult due to the lack of specific clinical symptoms. Niclosamide is the drug of choice for treatment of T. saginata and Taenia solium (pork tapeworm) infection; cure rates are approximately 90%. It is not absorbable and thus is nontoxic. Alternative treatments of taeniasis vary in the degree of safety.

Naegleria fowleri—commonly known as brain-eating amoeba—is single-celled and free-living and thrives in warm bodies of water. This parasite can cause a rare brain infection called meningoencephalitis, which causes severe brain inflammation. The amoeba also causes a whole host of other neurological symptoms and has a fatality rate approaching 100%. If water containing the amoeba enters the nose, the parasite can travel via the olfactory nerves, which are responsible for detecting odour molecules and transmitting them as signals to the brain. The parasite has been detected in South America and Asia but cases have also been reported in Australia, US and the UK. Naegleria fowleri infection is diagnosed based on microscopic examination of the fluid present the central nervous system, where active amoebae may be detected. Perhaps the most-agreed-upon medication for the treatment of N. fowleri infection is amphotericin B, which has been studied in vitro and also used in several case reports. Other anti-infectives which have been used in case reports include fluconazole, miconazole, miltefosine, azithromycin, and rifampin.

In some embodiments, the antiparasitic is chosen from the list comprising Chloroquine, Quinie, Mefloquine, Primaquine, Fansidar (Sulfadoxine and/or Pyrimethamine), Doxycycline, Atovaquone-proguanil, Artemether-lumefantrine.

In some embodiments, the antiparasitic is chosen from the list comprising Albendazole, Amphotericin B, Artemether-lumefantrine, Artesunate, Atovaquone, Atovaquone/proguanil, Azithromycin, Benznidazole, Bithionol, Chloroquine phosphate, Ciprofloxacin, Clarithromycin, Clindamycin, Clindamycin, Crotamiton, Dapsone, Dapsone+trimethoprim, Dapsone+Atovaquone, Dapsone+clindamycin, Dapsone+Pentamidine, Dapsone+Primaquine, Dapsone+pyrimethamine, Diethylcarbamazine, Diloxanide furoate, Doxycycline, Eflornithine, Eflornithine+nifurtimox, Fluconazole, Flucytosine, Fumagillin, Fumagillin+albendazole, Furazolidone, Iodoquinol, Ivermectin, Liposomal amphotericin B, Malathion, Mebendazole, Mefloquine, Meglumine antimoniate, Melarsoprol, Metronidazole, Miltefosine, Niclosamide, Nifurtimox, Nifurtimox/eflornithine, Nitazoxanide, Oxamniquine, Paromomycin, Paromomycin, Pentamidine, Permethrin, Praziquantel, Prednisone, Primaquine, Pyrantel, Pyrethrins with piperonyl butoxide, Pyrimethamine, Quinacrine, Quinidine gluconate, Quinine, Quinine dihydrochloride, Quinine sulfate, Rifampin, Sodium stibogluconate, Spinosad, Sulfadiazine, Suramin, Tetracycline, Tinidazole, Triclabendazole trimethoprim/sulfamethoxazole.

In some embodiments, the antiparasitic is chosen from the list comprising Amphotericin B, Azithromycin, Benznidazole, Bithionol, Chloroquine phosphate, Ciprofloxacin, Clarithromycin, Clindamycin, Clindamycin, Diethylcarbamazine, Diloxanide furoate, Doxycycline, Eflornithine, Fluconazole, Flucytosine, Fumagillin, Furazolidone, Iodoquinol, Ivermectin, Liposomal amphotericin B, Malathion, Mebendazole, Mefloquine, Meglumine antimoniate, Niclosamide, Nifurtimox, Nifurtimox/eflornithine, Prednisone, Primaquine, Pyrantel, Pyrimethamine, Quinine.

The infection to be treated using a composition or method according to the invention may be in a tissue and/or an organ in any part of the body of a subject.

In one embodiment, the infection is bacterial meningitis, such as an infection caused by any one or more of Neisseria meningitidis, Haemophilus influenzae, Escherichia coli, Streptococcus agalactiae, Streptococcus pneumoniae, Listeria monocytogenes.

In one embodiment, the infection is otitis media, such as acute otitis media, such as otitis media with effusion, such as an infection with Streptococcus pneumoniae.

In one embodiment, the infection is an eye infection, such as an infection with one or more of Staphylococcus spp., Streptococcus spp.

In one embodiment, the infection is a sinusitis, such as acute sinusitis, such as chronic sinusitis, such as an infection with one or more of Haemophilus influenzae, Moroxella catarrhalis, Streptococcus pneumoniae.

In one embodiment, the infection is an upper respiratory tract infection, such as an infection with one or more of Haemophilus influenzae, Streptococcus pyogenes.

In one embodiment, the infection is a pneumonia, such as an infection with one or more of Pseudomonas aeruginosa, Acinetobacter baumannii, Enterobacteriaceae, Haemophilus influenzae, Streptococcus pneumoniae.

In one embodiment, the infection is a skin infection, such as an infection with one or more of Pseudomonas aeruginosa, Staphylococcus aureus, Streptococcus pyogenes.

In one embodiment, the infection is a gastritis, such as an infection with Helicobacter pylori.

In one embodiment, the infection is a food poisoning, such as an infection with one or more of Campylobacter spp., Escherichia coli, Salmonella enterica, Shigella spp., Staphylococcus aureus, Listeria spp.

In one embodiment, the infection is a urinary tract infection, such as an infection with one or more of Escherichia coli, Klebsiella spp., Pseudomonas aeruginosa, Proteus spp., Enterococcus spp.

In one embodiment, the infection is a sexually transmitted disease, such as an infection with one or more of Chlamydia trachomatis, Neisseria gonorrhoeae, Haemophilus ducreyi.

In certain embodiments, the infection is one of more of an infection in the central nervous system; an aspergilloma; acute bacterial cholangitis; a catheter associated and/or complicated UTI.

The tissue penetration and distribution of an antimicrobial agent depend on various factors including drug characteristics such as molecular weight, protein binding, lipid solubility and degree of ionisation; target tissue characteristics such as membrane function and vascularisation of the tissue, and the presence or absence of inflammation. The skilled person appreciates the necessity of selecting an infection with antimicrobial under-exposure and that is focal enough to benefit from ultrasound targeting.

It should be noted that the treatment of focal locations can relieve stress on the immune system, which can trigger an immune reaction resulting in treatment of systemic disease.

In some embodiments, the pharmaceutical composition or method or system of the invention provides an increase in the therapeutic effect, compared to using the antimicrobial agent alone. In certain embodiments, the increase in the therapeutic effect is in the form of one or more of the following; improved uptake of the antimicrobial agent, reduced microbial density/count, reduced volume/area of infected area, improved quality of life, total eradication of the infection, improvement of overall survival, improvement of median survival, improvement of progression free survival

In certain embodiments, the infection to be treated using the composition or method according to the invention is related to an organ transplant. Solid organ transplantation is an effective life-sparing modality for thousands of patients worldwide with organ failure syndromes. In 2008, more than 29,000 solid organ transplant procedures were performed in the United States alone. Despite important advances in surgical technique and immunosuppressive regimens, substantial risks for post-transplantation infection remain, of which invasive fungal infections (IFIs) are among the most important. The most commonly reported IFIs among organ transplant recipients are invasive candidiasis, cryptococcosis, and invasive mold infections, such as aspergillosis and zygomycosis. The incidence of IFIs varies in frequency and specific etiology according to the type of organ transplant procedure and transplant center. An in-depth understanding of the overall burden of IFIs in this population is generally lacking. Organs that are transplanted include heart, pancreas, kidney, liver, lung, bone, and cornea.

The most common infection after transplantation is Pseudomonas infection. Pseudomonas is a genus of Gram-negative gammaproteobacteria, belonging to the family Pseudomonadaceae and containing 191 validly described species. The members of the genus demonstrate a great deal of metabolic diversity and are consequently able to colonise a wide range of niches. Their ease of culture in vitro and the availability of an increasing number of Pseudomonas strain genome sequences has made the genus an excellent focus for scientific research; the best studied species include P. aeruginosa in its role as an opportunistic human pathogen, the plant pathogen P. syringae, the soil bacterium P. putida, and the plant growth-promoting P. fluorescens, P. lini, P. migulae, and P. graminis Most Pseudomonas spp. are naturally resistant to penicillin and most related beta-lactam antibiotics, but a number are sensitive to piperacillin, imipenem, ticarcillin, or ciprofloxacin. Aminoglycosides such as tobramycin, gentamicin, and amikacin are other choices for therapy.

In one embodiment, the infection to be treated using the composition or method according to the invention is a prosthetic joint implant infection. Infections due to Gram-positive and Gram-negative pathogens associated with foreign implants or with intravascular catheters are very difficult to manage by antimicrobial therapy. Removal of implanted devices is often inevitable and has been standard clinical practice. The ability of pathogens to adhere to materials and promote biofilm formation is the most important feature of their pathogenicity.

In one embodiment, the infection to be treated using the composition or method according to the invention is an osteomyelitis, such as an infection by one or more of S aureus, Pseudomonas, Enterobacteriaceae. Bone is normally resistant to bacterial colonisation, but events such as trauma, surgery, the presence of foreign bodies, or the placement of prostheses may disrupt bony integrity and lead to the onset of bone infection. Osteomyelitis can also result from hematogenous spread after bacteremia. When prosthetic joints are associated with infection, microorganisms typically grow in biofilm. The normal treatment of osteomyelitis includes antibacterial therapy in combination with a surgical approach. Course of therapy is extended as penetration of drugs into the lesion is poor and often associate with biofilms formed on implanted metalwork.

The ATC treatment of the invention has a clear potential benefit in overcoming underexposure/pure penetration and addressing biofilm issues. Advantageously, the infection is focalised. ACT has potential advantages in
decreasing the need for surgical removal of prosthetic implant;
shortening the duration of treatment;
causing an earlier switch to oral therapy; and
shortening the hospital length of stay;
   and therefore also has a high potential cost-effectiveness impact.

In one embodiment, the infection to be treated using the composition or method according to the invention is a bacterial endocarditis, such as an infection with one or more of Staphylococci, Streptococci. Endocarditis is an infection of the endocardium i.e. inner lining of heart chambers and heart valves. It can be seen as an old problem in a new guise: Linked to underlying rheumatic heart disease in the pre-antibiotic and early antibiotic eras, prosthetic valve replacement, hemodialysis, venous catheters, immunosuppression now represent the principal risk factors. The average patient is older and frailer, with increasing comorbidities, and the mortality is high (10-20% in-hospital mortality). Long duration of intravenous (IV) antibacterial treatment is recommended. Bacterial endocarditis represents a significant burden for patients and hospitals alike with a median hospital length of stay of 43 days (French data), and average hospital charges in excess of $120,000 per patient (US data). Despite trends toward earlier diagnosis and surgical intervention, the 1-year mortality has not improved in over 2 decades.

The poor penetration within vegetations may be improved by ACT. Vascularisation present with or without inflammation—anatomy-pathology studies have dispelled the myth of a vascularisation of cardiac valves. There is potential for ACT to impact by:

decreasing the long hospital length of stays;

decreasing the long treatment durations;

causing an earlier switch to oral therapy; and

ultimately increase survival.

In one embodiment, the infection to be treated using the composition or method according to the invention is an acute bacterial prostatitis. Prostatitis is a common urologic disease seen in adult men, and is usually caused by the same bacteria that cause urinary tract infections. As many as 50% of men will experience an episode of prostatitis in their lifetime—2% to 3% of men will have bacterial prostatitis. Prostate biopsies (over 1 million each year in Europe) are a common cause. Prophylaxis represents standard of care, but still a high percentage of patients will develop post-biopsy infection. Prostate acid environment and lipidic epithelium lead to poor antibiotic exposure, and long treatment durations are required, typically six weeks.

In preferred embodiments, the infection to be treated using a composition or method according to the invention is selected from the group of endocarditis, prosthetic joint infections (PJI) (or other foreign body infections), osteomyelitis, prostatitis, ventriculitis, brain abscesses, aspergilloma or acute bacterial cholangitis. For each disease, depending on the type of infection (i.e. type of microorganism and its level of resistance to antimicrobial agents), the invention should be combined with the current Standard of Care antimicrobial agents.

In preferred embodiments, for treatment of endocarditis, the one or more antimicrobial agents is selected from the following list: penicillin, penicillin G, ceftriaxone, vancomycin, gentamicin, nafcillin, oxacillin, ampicillin, streptomycin, sulbactam and rifampin.

In preferred embodiments, for treatment of PJI, the one or more antimicrobial agents is selected from the following list: rifampin, nafcillin, cefazolin, geftriaxone, vancomycin, penicillin G, ampicillin, cefepime, meropenem, ertapenem, beta-lactam, ciprofloxacin and ceftriaxone.

In preferred embodiments, for treatment of osteomyelitis, the one or more antimicrobial agents is selected from the following list: Dalbavancin, clindamycin, rifampin, clindamycin, bactrim, linezolid, quinolone, fluconazole, doxycycline, cephalosporin, trimethoprim-sulfamethoxazole, levofloxacin, moxifloxacin, linezolid, minocycline, metronidazole clindamycin, penicillin, penicillin G, nafcillin, ampicillin, ampicillin-sulbactam and cefazolin.

### Subjects:

The subject to be treated may be a human or a non-human mammalian subject. The subject may be male or female. In some embodiments, the subject is an adult (i.e. 18 years of age or older). In certain embodiments, the subject is geriatric. In certain embodiments, the subject is not geriatric. In yet other embodiments, the subject is paediatric (i.e. less than 18 years of age). In some embodiments, the subject also has another pathology, such as a tumour. In other embodiments, the subject does not have a tumour. In some embodiments, the subject is an immune suppressed individual. In some embodiments, the subject has had an organ transplant. In some embodiments, the subject is a trauma patient, a burn patient, a patient on organ transplant drugs, a diabetic, a patient with AIDS.

### Administration Routes:

The cluster composition is preferably administered to said mammalian subject parenterally, preferably intravenously. The route of administration might also be selected from the intra-arterial, intramuscular, intraperitoneal, intratumoural or subcutaneous administration. An antimicrobial agent may be pre-, and/or co- and/or post administered to the cluster composition and may be a separate composition. In one embodiment, it may also be loaded into the microdroplet of the cluster composition, although how to practically carry this out is not yet solved. The antimicrobial agent is administered by a route suitable for the type of drug and the formulation form this is provided in. Typically, the route is selected from the group comprising, but not limited to, oral administration, intravenous (IV) administration, intramuscular (IM) administration, intrathecal administration, subcutaneous (SC) administration, sublingual administration, buccal administration, rectal administration, vaginal administration, administration by the ocular route, administration by the otic route, nasal administration, administration by inhalation, administration by nebulization, cutaneous administration, transdermal administration. The two compositions, i.e. the cluster composition (a) and the antimicrobial agent composition (b) may hence be administered via the same or via different routes of administration.

### Procedure:

The present invention can be used as a first-line or a second-line treatment, or any other kind of treatment.

It will be appreciated that the composition for use, the method for treatment, and/or the system or method for delivery of drugs, of the invention, may e.g. be employed as part of a multi-drug treatment regime. In one embodiment, the pharmaceutical composition for use according to the invention, includes the use of more than one antimicrobial agent. Furthermore, in one embodiment, several ACT treatments can be performed during the period of administrating the antimicrobial agents.

Hence, in one embodiment, more than one antimicrobial agent, such as 1 to 5 antimicrobial agents, are administered simultaneously or sequentially over a certain time span, such as over up to 3 hours, wherein at least one, such as 1 to 5, ACT treatments are performed during the same period. In one embodiment, the following ACT procedure is provided; intravenous administration of a cluster composition is followed by local ultrasound (US) insonation (activation) of the site of infection performed 3 consecutive times either immediately prior to or immediately after administration of antimicrobial agents.

Several therapeutic drugs can be used, and several ACT procedures can be applied during the treatment regime. In one embodiment, the ACT procedure is performed when the active therapeutic molecule displays maximum or close to maximum concentration in the blood after administration. Hence, the timing of the ACT treatment(s) may vary dependent upon the pharmacokinetics of the antimicrobial agent.

It will also be appreciated that the cluster composition may be used for the preparation of a subject for subsequent treatment with an antimicrobial agent.

In the treatment of serious infections for which rapid effect is essential, the dosage of the antimicrobial agent is of high importance. The higher the dosage of the antimicrobial agent that can be delivered to the site of infection, the higher the effect. In some embodiments, ACT is given close to when the antimicrobial agent is at its maximum serum concentration in a specified compartment of the body after (i.e. the site of infection) the drug has been administered.

The antimicrobial agent(s) are pre-, and/or co- and/or post administered to the cluster composition. In a preferred embodiment, an antimicrobial agent is administered after the administration of one of the at least one cluster compositions. Performing the ACT treatment, i.e. the administration of the cluster composition followed by the two step US procedure, before administration of the antimicrobial agent may give similar effect size as if the ACT treatment was initiated after administration of the antimicrobial agent (i.e. when the antimicrobial agent is in the blood stream). This may be beneficial in clinical practice, as the ACT treatment may be performed prior to starting the therapeutic administration and treatment. Hence, in one embodiment, an antimicrobial agent is administered after the cluster composition has been administered and activated in-vivo. In another embodiment, the cluster composition is administered either immediately prior to or immediately after administration of antimicrobial agent(s).

Hence, in one embodiment, the invention provides a pharmaceutical composition for use in a method of delivering an antimicrobial agent, wherein the method comprises the steps of:

(i) administering the pharmaceutical composition as defined in the first aspect to a mammalian subject with an infection; wherein at least one antimicrobial agent is pre-, and/or co- and/or post administered to the cluster composition, and before steps ii) to iii) or after any of steps ii) to iii);
(ii) optionally imaging the clusters of said pharmaceutical composition using ultrasound imaging to identify the region of interest for treatment within said subject;
(iii) activating a phase shift of the diffusible component of the second component of the cluster composition from step (i) by ultrasound irradiation of a region of interest within said subject, such that:
(a) the microbubbles of said clusters are enlarged by said diffusible component of step (iii) to give enlarged bubbles which are localised at said region of interest due to temporary blocking of the microcirculation at said region of interest by said enlarged bubbles; and
(b) facilitating extravasation of the antimicrobial agent(s) administered in step (i); and,
(iv) facilitating further extravasation of the antimicrobial agents administered in step (i) by further ultrasound irradiation.

Hence, the invention provides a microbubble/microdroplet cluster composition described above for use in a method of delivering an antimicrobial agent to a subject with an infection, wherein the method comprises the steps of:

(i) administering at least one antimicrobial agent selected from the group of antibiotics, antifungals, antivirals, antiparasitics, or combinations thereof to the subject;
(ii) administering the microbubble/microdroplet cluster composition to the subject;
wherein the at least one antimicrobial agent is pre-, and/or co- and/or post administered to the cluster composition;
(iii) activating a phase shift of the diffusible component of the second component of the cluster composition from step (ii) by ultrasound irradiation of a region of interest within said subject;
(iv) facilitating extravasation of the antimicrobial agents administered in step (i) by further ultrasound insonation.

The duration of therapy may be guided by the severity and site of the infection and the subject's clinical and microbial progress. Treatments may be performed as often and as many times as necessary, depending on the treatment regime. The ultrasound procedure, steps (iii) and (iv) are performed as disclosed above.

The invention further provides a method of delivering at least one antimicrobial agent to a mammalian subject, comprising the steps of:

(i) administering the pharmaceutical composition as defined in the first aspect to a mammalian subject;
(ii) optionally imaging the microbubbles of said pharmaceutical composition using ultrasound imaging to identify the region of interest for treatment within said subject;
(iii) activating a phase shift of the diffusible component of the second component of the cluster composition from step (i) by ultrasound irradiation of a region of interest within said subject, such that:
(a) the microbubbles of said clusters are enlarged by said diffusible component of step (iii) to give enlarged bubbles which are localised at said region of interest due to temporary blocking of the microcirculation at said region of interest by said enlarged bubbles; and
(b) said activation of step (iii) facilitates extravasation of the antimicrobial agent(s) administered in step (i); and,
(iv) facilitating further extravasation of the antimicrobial agents administered in step (i) by further ultrasound irradiation.

As provided for the first aspect, the mammalian subject is e.g. a subject having an infection.

In a further aspect, the invention provides a method of treatment of an infection of a mammalian subject, comprising the step of administering to the subject a pharmaceutical composition comprising:

(a) a cluster composition which comprises a suspension of clusters in an aqueous biocompatible medium, where said clusters have a mean diameter in the range 1 to 10 μm, and a circularity <0.9 and comprises:
(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions;
(b) an antimicrobial agent selected from the group of antibiotics, antifungals, antivirals, antiparasitics, or combinations thereof, provided as a separate composition to (a).

In yet a further aspect, the invention relates to the use of a cluster composition for preparation of a subject for subsequent treatment with an antimicrobial agent, said cluster composition comprising a suspension of clusters in an aqueous biocompatible medium, where said clusters have a mean diameter in the range 1 to 10 μm, and a circularity <0.9 and comprises:

(i) a first component which comprises a gas microbubble and first stabiliser to stabilise said microbubble; and
(ii) a second component which comprises a microdroplet comprising an oil phase and second stabiliser to stabilise said microdroplet, where the oil comprises a diffusible component capable of diffusing into said gas microbubble so as to at least transiently increase the size thereof;
where the microbubbles and microdroplets of said first and second components have opposite surface charges and form said clusters via attractive electrostatic interactions. Such use comprises the ultrasound procedure, steps (iii) and (iv), as disclosed above.

The embodiments and features described in the context of one aspect, e.g. for the aspect directed to the composition for use, also apply to the other aspects of the invention.

The invention shall not be limited to the shown embodiments and examples. While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the scope of the present invention. It is to be understood that various alternatives to the embodiments described herein can be employed in practicing the disclosure. Further, it is contemplated that the appended claims will cover such modifications and variations that fall within the true scope of the invention.

It is to be understood that every embodiment of the disclosure can optionally be combined with any one or more of the other embodiments described herein.

It is to be understood that each component, compound, or parameter disclosed herein is to be interpreted as being disclosed for use alone or in combination with one or more of each and every other component, compound, or parameter disclosed herein. It is further to be understood that each amount/value or range of amounts/values for each component, compound, or parameter disclosed herein is to be interpreted as also being disclosed in combination with each amount/value or range of amounts/values disclosed for any other component(s), compound(s), or parameter(s) disclosed herein, and that any combination of amounts/values or ranges of amounts/values for two or more component(s), compound(s), or parameter(s) disclosed herein are thus also disclosed in combination with each other for the purposes of this description. Any and all features described herein and combinations of such features are included within the scope of the present invention provided that the features are not mutually inconsistent.

It is to be understood that each lower limit of each range disclosed herein is to be interpreted as disclosed in combination with each upper limit of each range disclosed herein for the same component, compound, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, or parameter disclosed in the description or an example is to be interpreted as a disclosure of either a lower or an upper limit of a range and thus can be combined with any other lower or upper limit or a range or specific amount/value for the same component, compound, or parameter disclosed elsewhere in the application to form a range for that component, compound, or parameter.

### EXAMPLES

### Example 1. Cluster Preparation, Analytical Tools and Basic Characteristics

Reference is made to application WO2015/047103, and particularly to the Examples of this, the contents of which are incorporated herein by reference, providing descriptions of analytical methodologies for characterisation of the clusters compositions, results from use of the clusters, etc.

In the following examples the first component is designated C1, the second component is designated C2 and the cluster composition, i.e. the composition resulting from a combination of the first and second components, is designated DP (drug product).

Example 1 provides descriptions of analytical methodologies for characterisation and quantitation of microbubble/microdroplet clusters in DP, and explains relevant responses and attributes including concentration, size and circularity. It also provides details on analytical methodology for characterisation and quantification of activated bubble size and concentration. In addition, data on cluster stability after preparation are presented, as is a comparison of characteristics for pre-mixed vs. co-injected DP.

It also details engineering steps for controlled manipulations of cluster content and size in DP.

Example 1 further provides results from in-vivo studies elucidating effects of cluster characteristics on product efficacy as the ability to deposit large, activated bubbles in the microcirculation. It further analyses these data and concludes that clusters with a mean size between 3 to 10 μm, defined by a circularity of less than 0.9, are contributing to the efficacy of the cluster composition.

The microbubble/microdroplet clusters formed upon combining C1 and C2, i.e. present in DP, are crucial to the critical quality attributes of the composition, i.e. its functionality for delivery of drugs. Hence, analytical methodology to characterize and control the clusters formed with regards to concentration and size, is an imperative tool to assess the current invention as well as for medicinal Quality Control (QC). We have identified three different analytical tools that can be applied for this purpose; Coulter counting, Flow Particle Image Analysis (FPIA) and Microscopy/Image analysis.

In addition to these techniques, applied for characterisation of the clusters in the cluster composition, analytical methodology has been developed to study the activation of the clusters in vitro, i.e. the generation of large, activated bubbles upon ultrasound irradiation. This methodology; “Sonometry” is detailed in E1-6 of WO2015/047103. Primary report responses from the Sonometry analysis are number and volume of activated bubbles and their size distribution, both vs. time after activation. Activation responses may also be explored by Microscopy/Image analysis as detailed in E1-5 of WO2015/047103.

### Components and Compositions:

The first component (C1) in the compositions investigated in the included example consisted of per-fluorobutane (PFB) microbubbles stabilised by a hydrogenated egg phosphatidyl serine-sodium (HEPS-Na) membrane and embedded in lyophilised sucrose. HEPS-Na carries a negatively charged head group with an ensuing negative surface charge of the microbubbles. Each vial of C1 contains approximately 16 μL or 2·10⁹ microbubbles, with a mean diameter of approximately 2.0 μm.

The second component (C2) in the compositions investigated in this example consisted of perfluoromethyl-cyclopentane (pFMCP) microdroplets stabilised by a 1,2-Distearoyl-sn-glycerol-3-phosphocholine (DSPC) membrane with 3% mol/mol stearylamine (SA) added to provide a positive surface charge. The microdroplets in the C2 were dispersed in 5 mM TRIS buffer. The standard formulation of C2 investigated in these studies contains approximately 4 μL or 0.8·10⁹ microdroplets per mL, with a mean diameter of approximately 1.8 μm.

In some cases, to elucidate effects on cluster characteristics, a variety of formulation variables such as SA content, microdroplet size, microdroplet concentration, TRIS concentration and pH was varied in a controlled manner. In case such samples have been used, these aspects are detailed in the text.

The cluster composition (DP) was prepared aseptically by reconstituting a vial of C1 with 2 mL of C2 followed by 30 seconds of manual homogenisation. 2 mL was withdrawn from a vial of C2 using a sterile, single use syringe and needle. The content of the syringe was added through the stopper of a vial of C1 and the resulting DP was homogenised.

As shown in WO2015/047103, the first and second components, i.e. the microbubble formulation and the microdroplet formulation, can be varied. E.g. as shown in tables 9 and 10 of WO2015/047103 both the gas and the stabilising membrane of the first component can be varied, to prepare clusters with suitable properties, expected to be useful in treatment according to the invention.

### Stability of Clusters in the Cluster Composition During Analysis:

The clusters in the DP are formed and kept by the electrostatic attraction between the microbubbles and the microdroplets. These forces are finite and the clusters may break up after formation through various routes/influences such as mechanical stress or thermal (Brownian) motion. For precise and accurate characterisation, it is important that the clusters remain stable during the time of analysis. This stability has been investigated with all the methodologies described above. To evaluate stability, 3 to 5 analyses where repeated on a single DP sample covering a timespan of >5 minutes. No significant change in neither concentration nor size has been observed cross these replicates, proving that the microbubbles, microdroplets and clusters are stable for >5 minutes under the analytical conditions stated, i.e. after dilution in PBS or water and under continuous homogenisation (stirring).

### Formulation Aspects:

A number of different formulation aspects can be explored for controlling the cluster content and size in the DP and for targeting optimal properties. Parameters that can be used to engineer cluster content and size distribution include, but are not limited to; the difference in surface charge between the microbubbles and the microdroplets (e.g. SA %: the microdroplet size of C2: the pH: the concentration of TRIS in C2: and the concentration of microbubbles and microdroplets. In addition, chemical degradation of the components, e.g. during prolonged storage at high temperatures, may influence the ability of C1 and C2 to form clusters during preparation of the DP.

From in-vitro characterisation of 15 different compositions, as reported in WO2015/047103, several important correlations that elucidate the nature and characteristics of the system can be extracted. We found that the size of the clusters formed is also strongly connected to the Reactivity of the system. Only small clusters (i.e. 1-5 um) and medium sized clusters (i.e. 5-10 μm) are formed at relatively low levels of Reactivity (e.g. <20%). With increasing Reactivity, larger clusters start to form; at R>approx. 20%, 10-20 μm clusters start to form and at R>approx. 50%, 20-40 μm clusters start to form. When larger clusters form, it is at the expense of smaller and medium sized clusters; we found a clear optimum in content vs. Reactivity for cluster concentration 1-5 μm and 5-10 μm. We found that formation of larger clusters is detrimental to the efficacy of the composition and that the clustering potential must be balanced accordingly.

Based on applicant's experiments, and the results shown in Tables 5 and 6 of WO2015/047103, the efficacy (linear enhancement (GS)) of the cluster composition is at least based on the cluster mean size and the concentration of clusters (million/ml). The results reported there are from a multivariate, principal component analysis (PCA) of the contribution of clusters in various size classes to the linear enhancement in the ultrasound signal from dog myocardium (Grey Scale units) upon i.v. administration of the cluster composition and activation in the left ventricle, please see Example 2 of WO2015/047103. The PCA was performed on data for 30 samples detailed in Tables 5 and 6 of this. The results demonstrate that small and medium sized clusters (<10 μm) contribute significantly to the efficacy of the cluster composition whereas larger clusters (>10 μm) do not. These results and conclusion also apply for the current invention. The cluster size distribution is important, and the mean size should be in the range of 3-10 μm, and preferably 4-9 μm, more preferably 5-7 μm.

The cluster concentration and mean diameter of the cluster composition, prepared according to Example 1, was analysed and found to have a cluster concentration of about 40-44 million per mL and with a cluster mean diameter of about 5.8-6.2 μm, for several hours. The results are shown in Table 1 below and are consistent with the results of Table 6 of WO2015/047103. The data of Table 1 shows that the prepared cluster composition has an acceptable stability, and that an optimal concentration of cluster size can be achieved.

TABLE 1

| Time | Cluster Concentration | Cluster Mean Diameter |
|---|---|---|
| (hours) | (millions/mL) | (μm) |
| 0 h | 44 ± 2 | 6.0 ± 0.2 |
| 1 h | 43 ± 1 | 5.8 ± 0.2 |
| 2 h | 44 ± 5 | 6.2 ± 0.1 |
| 3 h | 40 ± 1 | 6.0 ± 0.2 |

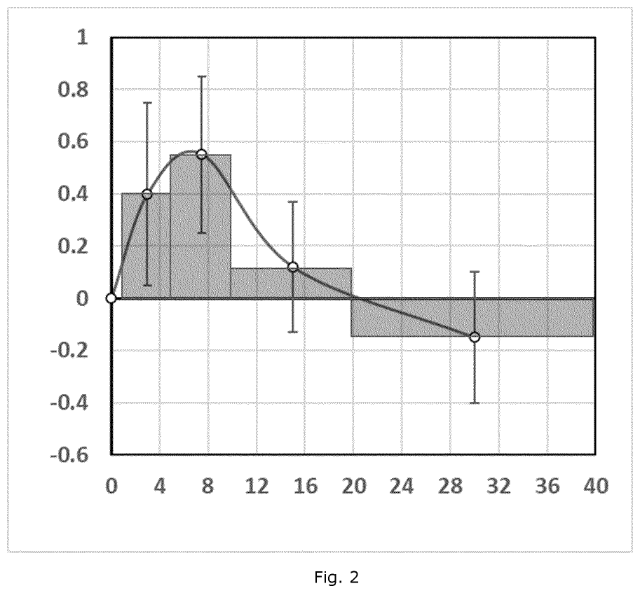

The size of the clusters affects the efficacy. FIG. 2 shows a visualisation of cluster size versus product efficacy, demonstrating that clusters having a mean diameter in the range of 3 to 10 μm are optimal the efficacy of the cluster composition. In FIG. 2 the Y-axis shows the calculated correlation coefficient, i.e. the contribution to myocardial enhancement for cluster size classes (X-variables) 1-5 μm, 5 to 10 μm, 10 to 20 μm and 20 to 40 μm. FIG. 2 is an alternative visualisation of FIG. 12 (left side) of WO2015/047103 and is based on the data provided in Table 2 below.

TABLE 2

| Channel Group | Mean Channel Diameter | Efficacy Coefficient |
|---|---|---|
| 1 to 5 μm | 3 | 0.4 |
| 5 to 10 μm | 7.5 | 0.55 |
| 10 to 20 μm | 15 | 0.12 |
| 20 to 40 μm | 30 | −0.15 |

Applying the concept of the present invention, i.e. by preparing a cluster composition from C1 and C2 prior to administration, hence forming microbubble/microdroplet clusters, opposed to co-injection of the two components as taught by WO/9953963, enable a >10-fold increase in efficacy. The formation of microbubble/microdroplet clusters upon combination of the first component and second component, and administering these pre-made clusters, is a pre-requisite for its intended functionality in-vivo.

### Example 2. ACT Induced, Localized Delivery of Drug Mimicking Molecules and Nanoparticles

To demonstrate that the ACT concept can enhance localized delivery of antimicrobial agents, such as antibiotic agents, two in-vivo studies were performed. In the first of these (Study 1), tissue specific delivery of a drug mimicking chromophore (Evans Blue) was investigated in a mouse model. In the second (Study 2), localized delivery of a drug mimicking nanoparticle across the Blood Brain Barrier was investigated in a mouse model.

### Study 1—Tissue Specific Delivery of Evans Blue

The tissue specific uptake of Evans Blue (EB, a fluorescent dye) has been investigated in a mouse model. Under physiologic conditions, the endothelium is impermeable to albumin, and Evans blue bound albumin remains confined within blood vessels. Thus, Evans blue is often used as a model compound in drug delivery studies [Bohmer et al., J Controlled Release, 148, Issue 1, 2010, pp. 18-24].

### Materials and Methods:

Female Balb/c nude mice were used. Before treatment, the mice were administered surgical anesthesia by subcutaneous injection of a mix of Fentanyl (0.05 mg/kg), Midazolam (5 mg/kg), and Medetomidine (0.5 mg/kg). An intravenous cannula (BD Neoflon™ 24 GA) was placed in the tail vein. Patency was verified by injection of a slight amount of 0.9% sodium chloride for injection after which a small amount of heparin (10 U/ml) was injection to prevent clotting. The hub of the cannula was filled with 0.9% sodium chloride for injection to eliminate any dead space and closed with a cap. The cannula was secured to the tail with surgical tape.

Evans Blue solution (50 mg EB/kg) was injected i.v. followed immediately by 2 ml/kg of the cluster dispersion as detailed in Example 1. Two groups where compared (N=3 for each group): 1) EB+cluster dispersion and 2) EB+cluster dispersion+Activation and Enhancement ultrasound insonation.

The left hind limb of the mouse was placed in a water bath with two US transducers poised for insonation of the left thigh. Ultrasound Activation insonation was provided by a VScan clinical ultrasound scanner (GE Healthcare) with a 2 MHz probe for 45 seconds (MI=0.8) starting from the injection time. This was immediately followed by the Enhancement step comprising 5 minutes 500 kHz ultrasound insonation (MI=0.2) using a single element transducer (Imasonic SAS). Thirty minutes after treatment the animals were sacrificed, tissue samples; thigh muscle from the treated (left) leg and thigh muscle from the contra lateral untreated leg, were harvested and Evans Blue content extracted and quantified. The concentration in the treated thigh muscle was divided by the concentration in the untreated thigh muscle for each animal (matched pair) to provide a dimensionless ratio of the increased uptake in the treated muscle. A one-way ANOVA was applied to the data.

### Results:

For the animals which did not receive US insonation, the average±SD EB ratio of the left (treated) to right (untreated) was 1.1±0.2 and not significantly different. On the other hand, for the animals which received US insonation, the average±SD EB ratio of the left (treated) to right (untreated) was 2.0±0.3, demonstrating a significant (p<0.05) and about 100% increase in the uptake of EB upon ACT treatment.

### Study 2—Localized Delivery of Drug Mimicking Nanoparticles to Brain Tissue.

The delivery of a drug mimicking nanoparticle agent to specific locations within the brain parenchyma has been investigated in a mouse model. Models investigating delivery of therapeutic agents across the BBB and into brain tissue are hence often used as “the ultimate test” when evaluating various concepts for drug delivery.

### Materials and Methods:

The ACT cluster composition investigated was as detailed in Example 1.

The nanoparticles investigated were core-crosslinked polymeric micelles (CCPMs) from Cristal Therapeutics (Maastricht, The Netherlands). These CCPMs are 70 nm in diameter, labelled with rhodamine B Cy7 for imaging purposes, and the formulation contained 44 mg/ml polymer and 40 nmol/ml Cy7.

The extravasation of CCPM in healthy mouse brains was measured using near infrared fluorescence (NIRF) imaging and the micro-distribution of the CCPM in brain sections was imaged by confocal laser scanning microscopy (CLSM).

Thirteen female albino BL6 mice, purchased at 6-8 weeks of age (Janvier labs, France), were housed in groups of five in individually ventilated cages under conditions free of specific pathogens. Cages were enriched with housing, nesting material and gnaw sticks, and were kept in a controlled environment (20-23° C., humidity of 50-60%) at a 12-hour night/day cycle. Animals had free access to food and sterile water. All experimental procedures were approved by the Norwegian Food Safety Authority.

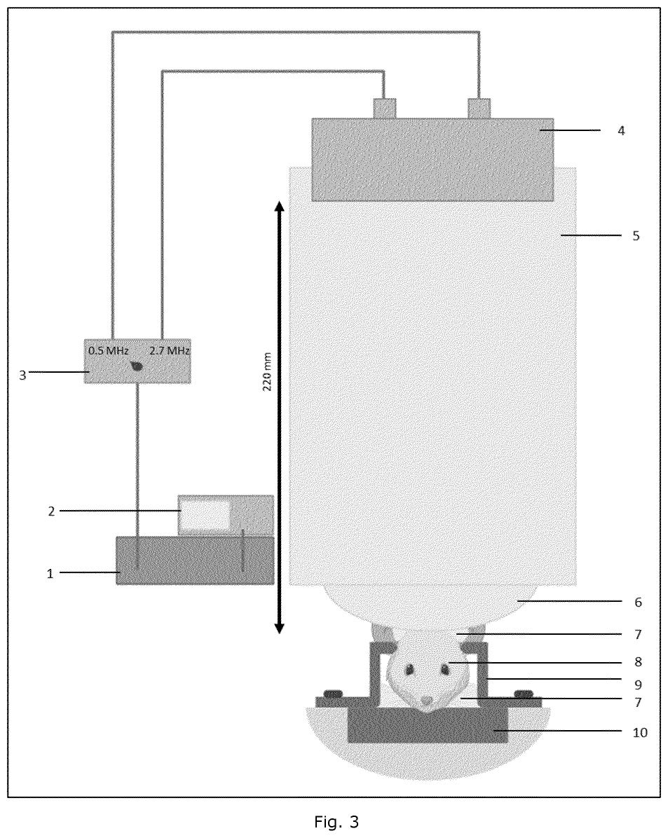

Illustration of the ultrasound set-up is shown in FIG. 3. A custom built dual frequency transducer (centre frequency 0.5 MHz and 2.7 MHz) [Andersen et al., A Harmonic Dual-Frequency Transducer for Acoustic Cluster Therapy, Ultrasound Med Biol 2019 September; 45(9): 2381-2390] was mounted on top of a custom made cone filled with degassed water. The transducer has a diameter of 42 mm and the −3 dB width of the beam profile of the 0.5 and 2.7 MHz were 16 and 6 mm at a distance of 220 mm from the transducer surface. Signals were generated with a signal generator (33500B, Agilent Technologies, USA) and amplified with a 50 dB RF amplifier (2100 L, E&I, USA). The amplifier is connected to the switch box which allows for switch from the Activation to the Enhancement US fields. The bottom of the cone was covered with an optically and acoustically transparent plastic foil (Jula Norge AS, Norway), forming a bag. The animal was positioned in prone position on top of an acoustically absorbing material (Aptflex F28, Precision Acoustics, UK), with ultrasound gel for coupling between the acoustic absorber, the animals head and the acoustically transparent foil.

The ACT procedure used comprised an activation and an enhancement step. The attenuation through the murine skull was measured to be approximately 21±17% and 42±21% for the 0.5 MHz and 2.7 MHz frequencies, respectively. These numbers where used to calculate the in situ acoustic pressures/Mls. The following ultrasound parameters were used for each step:
Activation: Centre frequency of 2.7 MHz, average in situ acoustic pressure of corresponding to mechanical index (MI) of 0.18, 8 cycles pulse length, pulse repetition frequency of 1 kHz and insonation time of 60 s.
Enhancement: Centre frequency of 0.5 MHz, average in situ acoustic pressure corresponding to MI of 0.15, 4 cycles pulse length, pulse repetition frequency of 1 kHz and insonation time of 300 s.

One round of ACT consisted of a bolus intravenous injection of 2 mL/kg of cluster composition prior to the 360 s insonation. Each animal received 3 rounds of ACT, resulting in a total of 75 μl ACT formulation and 18 min ultrasound. CCPM was injected i.v. immediately prior to the first ACT procedure.

Animals were anaesthetized using 2% isoflurane in medical air (78%) and oxygen (20%) (Baxter, USA) after which their lateral tail vein was cannulated. Hair was removed with a hair trimmer and depilatory cream (Veet, Canada). During the ACT procedure, animals were anaesthetized using 1.5-2% isoflurane in medical air. Respiration rate was monitored using a pressure sensitive probe (SA instruments, USA) and body temperature was maintained with external heating. Each animal received 3 ACT rounds directly after injection of CCPM. Control animals were handled in the same way as the ACT receiving animals but received 3 times a 50 μl saline injection instead of the cluster composition with 6 minutes interval.

Two timepoints were investigated: 1 and 24 hours after ended ACT treatment. At these timepoints, animals were euthanized by an intraperitoneal injection of pentobarbital (200 μl) and kept under anaesthesia until their breathing halted. Thereafter they were transcardially perfused with 30 ml of PBS after which the brain was excised and imaged with the NIRF imager. Groups were; control/1 hour N=3, control/24 hours N=3, ACT/1 hour N=5 and ACT/24 hours N=2.

Excised brains were placed in a NIRF imager (Pearl Impulse Imager, LI-COR Biosciences Ltd., USA) to assess accumulation of CCPM® in the brain. Brains were excited at 785 nm and fluorescence emission was detected at 820 nm. Images were analysed with ImageJ (ImageJ 1.51j, USA). A Region of Interest (ROI) was drawn around the brain and the total fluorescence intensity of the brain was acquired and normalized to the wet weight of the brain. A standard curve was used to convert the total fluorescence intensity to the percentage of the injected dose per gram of brain tissue (% ID/g). Results were plotted per timepoint and treatment group.

For confocal microscopy, excised brains were mounted transversely on a piece of cork with Optimum Cutting Temperature Tissue Tek (Sakura, The Netherlands) before submerging the sample slowly in liquid nitrogen. Of the frozen brains, the first 500 μm from the top was removed after which 5×10 μm thick sections and 5×25 μm thick sections were cut transversely. This was repeated every 800 μm throughout the brain.

### Results:

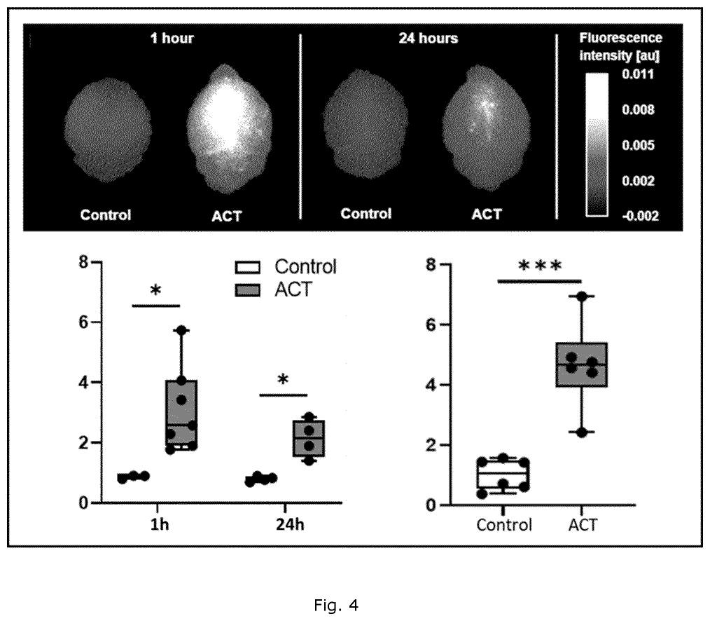

To study whether the increased permeability would facilitate extravasation of CCPMs, excised brains were imaged in a NIRF imager. Representative NIRF-images of controls and animals injected with the nanoparticles are shown in FIG. 4 (upper panels). As can be noted, clear accumulation could be observed in brains which received ACT opposed to the control brains.

Quantitative analysis of the NIRF-images revealed a statistically significant increase in accumulation (% ID/g) between the ACT and control animals at both timepoints (FIG. 4, left lower panel). Vs. control, with ACT the median % ID/g increased from 0.9% ID/g to 2.6% ID/g 1 hour and from 0.8% ID/g to 2.2% ID/g 24 hours after ACT. Respectively, a 290 and 280% increase in % ID/g was observed.

To verify the increased accumulation of the CCPM in brain tissue after ACT treatment, and to study the location of CCPM with respect to blood vessels, brain sections were imaged by CLSM. Tilescans of ACT-treated brains showed several ‘clouds’ of fluorescence which were not observed in brains of control animals. 24 hours post ACT, tilescans of ACT-treated brains showed similar cloud patterns as the 1 hour treatment group. From thresholded tilescans of both control and ACT-treated animals, the number of pixels representing CCPM were extracted and normalized by the size of the ROI used to outline the hemispheres. As can be noted from FIG. 4 (right lower panel), a clear and statistically significant 470% increase can be observed in the 1 h post ACT-treated sections compared to the control brains.

High magnification CLSM images at different locations in both the control brains and the ACT-treated brains were acquired to study the location of the CCPM with respect to the blood vessels. In ACT-treated brains, CCPM had clearly extravasated whereas in control brains CCPM were mainly observed intravascularly or minimally displaced from the blood vessel staining.

### Conclusions for Example 2:

ACT induced a 100% increase in the uptake of the drug mimicking molecule Evans Blue in US insonated tissue. Furthermore, ACT clearly increased the permeability of the BBB for large nanoparticle constructs like the 70 nm CCPM compound investigated, when applying the two-step insonation approach of the current invention. ACT resulted in a more than 200% increase in the extravasation to the brain parenchyma, localised to the insonated region of interest. These two studies hence demonstrate the ability of ACT to locally increase the concentration of co-administered therapeutic agents in a targeted tissue compartment and indirectly that the concept will enhance the therapeutic benefit of antimicrobial agents, such as antibiotics, Cmax-dependent such in particular.

### Example 3. Evaluation of ACT in the Levofloxacin Treatment of a Staphylococcal Thigh Infection in Mice

The following study is ongoing for the investigation of the benefit of ACT for enhancing antimicrobial effects of Levofloxacin (LEV) for treatment of Staphylococcus aureus thigh infection in mice. LEV is a broad spectrum, Cmax-dependent antibiotic for treatment of a variety of infections, including but not limited to acute bacterial sinusitis, pneumonia, H. pylori, urinary tract infections, chronic prostatitis, and gastroenteritis.

### Experimental Approach:

An established experimental model of staphylococcal thigh infection in a neutropenic thigh infection model was used and neutropenia will be induced with cyclophosphamide. The thigh infection model provides a sensitive experimental system for initial studies of antimicrobial efficacy in a mammalian system. This model is the most standardised for the evaluation of antimicrobial-microbial interactions combined with antimicrobial pharmacokinetics assessed in serum and tissues samples.

The challenge isolate was methicillin susceptible strain of Staphylococcus aureus is and both thighs were inoculated. Treatment was initiated 2 h after inoculation, thighs was harvested after 26 h and the bacterial density (colony forming units, CFU) in the excised tissue was determined. An initial dose finding study was performed to define the LEV ED₅₀ (the dose that induces a half maximal decline in bacterial density). Within the study, LEV was be dosed intraperitoneal at 20 mg/kg.

### Experimental Groups:

To demonstrate that ACT with LEV is significantly better than LEV alone, three groups was evaluated: 1) Vehicle control (saline), 2) LEV alone and 3) LEV+ACT (see ACT Procedure below).

### ACT Procedure:

The cluster composition investigated was as detailed in Example 1. The ultrasound apparatus for application of the ACT Sonoporation procedure was as detailed in Example 2/Study 2 with US focused on the site of infection. The cluster composition was administered intravenously at 2 mL/kg and followed by local ultrasound (US) insonation of the site of infection with 45 second of US Activation field (2.7 MHz, MI 0.3), followed with 5 minutes US Enhancement field (500 kHz, MI 0.2). The procedure was performed 3 consecutive times, starting immediately 10-15 minutes after administration of LEV.

The bacterial density in the thighs harvested from the various groups was determined by growing a diluted sample of the excised tissue, homogenized in sterile PBS, on a plate with growth medium and counting the number of CFU.

A number of experiments of the same design are planned for in order to build numbers for adequate statistical power.

### Results (Interim):

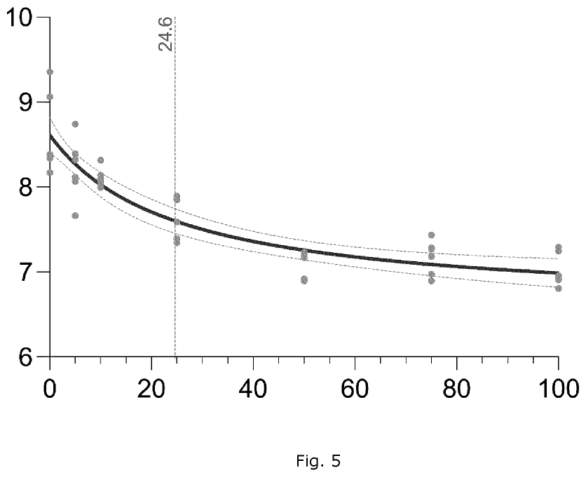

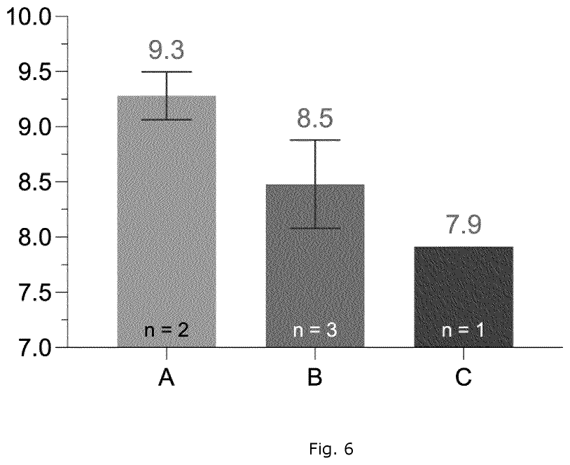

FIG. 5 shows the results from the dose-response of LEV in the investigated model. As can be noted ED₅₀ is estimated to approx. 24 mg/kg, hence, a dose of 20 mg/kg was investigated in the ACT treatment study. FIG. 6 shows interim results for the three groups investigated. Compared to saline control, a LEV dose of 20 mg/kg, treatment inhibits bacterial growth 0.8 log units (approx. 6-fold). However, combining ACT sonoporation with the same dose of LEV, further inhibits bacterial growth to a total of 1.4 log (approx. 25-fold).

Even though the number of data points are low, and the observed differences are hence not statistically significant, the interim findings from the study clearly indicate that the ACT procedure as described in the current invention enhances the therapeutic effect of antimicrobial agents for treatment of infections.

### Example 4 (Prospective). Evaluation of ACT in the Daptomycin Treatment of a Staphylococcal Thigh Infection in Mice

The following study is planned for the investigation of the benefit of ACT for enhancing antimicrobial effects of Daptomycin (DPT), a standard, Cmax-dependent antibiotic for Gram-positive infections, for treatment of Staphylococcus aureus thigh infection in mice.

### Methods:

Animals (CD-1 mice) will be rendered neutropenic by administration of two intraperitoneal injections of cyclophosphamide monohydrate (CPA) at day −4 (150 mg/kg, i.p.) and −1 (100 mg/kg, i.p.) (0=day of infection). The day of infection animals will be intramuscularly infected in the right thigh with 100 μL of the bacterial suspension of Staphylococcus aureus Xen29 (challenge: ˜10⁶ CFU/thigh). Start of treatment will be 2 hours post infection (0 hours). Vehicle and DPT will be subcutaneously administered. The dose of DPT will be selected as the “first not effective dose”, determined in a dose-range study prior to the ACT therapy study.

Three treatment groups will be investigated (N=6 for each group): 1) Vehicle control (saline), 2) DPT alone and 3) DPT+ACT.

Animals will be sacrificed after 24 hours and thighs collected and processed to obtain bacterial load determination.

### ACT Procedure:

The cluster composition investigated will be as detailed in Example 1. The ultrasound apparatus for application of the ACT Sonoporation procedure will be as detailed in Example 2/Study 2 with US focused on the site of infection. The cluster composition will be administered intravenously at 2 mL/kg and followed by local ultrasound (US) insonation of the site of infection with 45 second of US Activation field (2.7 MHz, MI 0.3), followed with 5 minutes US Enhancement field (500 kHz, MI 0.2). The procedure will be performed 3 consecutive times, starting immediately 10-15 minutes after administration of DPT.

### Results (Prospective):

The results will demonstrate that treatment with DPT alone shows no significant therapeutic effect vs. vehicle control (i.e. no significant difference in c.f.u.), but that ACT in combination with DPT shows a market and significant therapeutic effect, with more than a 10-fold reduction in c.f.u. vs. DPT alone.

### Example 5 (Prospective). Evaluation of ACT in the Rifampicin Treatment of a Staphylococcal Human Foreign Body Infection Model in Mice

The following study is planned for the investigation of the benefit of ACT for enhancing antimicrobial effects of Rifampicin (RIF) for treatment of Staphylococcus aureus Human Foreign Body infection in mice. RIF is a standard, Cmax-dependent antibiotic for treatment of several types of bacterial infections, including tuberculosis, Mycobacterium avium complex, leprosy, and Legionnaires' disease,

### Methods:

On the day of infection, animals (CD-1 mice) will be anaesthetized and surgically prepared. A subcutaneous pocket will be made on the right flank and a polyethylene catheter (0.5 cm of length) will be inserted. 100 μL/implant of the bacterial suspension of Staphylococcus aureus Xen29 (challenge: ˜10⁶ CFU/implant) will be injected in the catheter immediately after suture of the pocket. Start of treatment will be 2 hours post infection (0 h). Vehicle and RIF will be administered subcutaneously. The dose of RIF will be selected as the “first not effective dose”, determined in a dose-range study prior to the ACT therapy study. Treatments will be administered daily for 4 days.

Three treatment groups will be investigated (N=6 for each group): 1) Vehicle control (saline), 2) RIF alone and 3) RIF+ACT.

Animals will be sacrificed after 5 days of infection (24 hours from the last treatment) and implants collected and processed to determine bacterial load (CFU/implant).

### ACT Procedure:

The cluster composition investigated will be as detailed in Example 1. The ultrasound apparatus for application of the ACT Sonoporation procedure will be as detailed in Example 2/Study 2 with US focused on the site of infection. The cluster composition will be administered intravenously at 2 mL/kg and followed by local ultrasound (US) insonation of the site of infection with 45 second of US Activation field (2.7 MHz, MI 0.3), followed with 5 minutes US Enhancement field (500 kHz, MI 0.2). The procedure will be performed 3 consecutive times, starting immediately 10-15 minutes after administration of RIF.

### Results (Prospective):

The results will demonstrate that treatment with RIF alone shows no significant therapeutic effect vs. vehicle control (i.e. no significant difference in CFU), but that ACT in combination with RIF shows a market and significant therapeutic effect, with more than a 10-fold reduction in CFU vs. RIF alone.

### Example 6 (Prospective). Manufacture of Cluster Compositions with Various Microbubble and Microdroplet Components

In order to show that the invention is applicable for a variety of chemical compositions of the first and second components (C1 and C2), several formulations may be manufactured or sourced commercially and explored for the in-vitro attributes of the resulting cluster composition.

### C1 Examples

The commercially available microbubble US imaging agents Sonovue (Bracco Spa, Italy) and Micromarker (VisualSonics Inc., USA) may be sourced and used as C1 components. Sonovue is a sulphur hexafluoride microbubble stabilized with a membrane of distearoylphosphatidylcholine, dipalmitoylphosphatidylglycerol sodium, palmitic acid and PEG4000, and presented in a lyophilized form to be reconstituted with 5 mL of aqueous matrix. Micromarker is a perfluorobutane/nitrogen microbubble stabilized with phospholipids, polyethylenglycol and fatty acid, and presented in a lyophilized form for reconstitution with 0.7 mL of aqueous matrix.

### C2 Examples

Microdroplet (C2) components with diffusible components; perfluorodimethyl-cyclobutane, 2-(trifluoromethyl)perfluoropentane and perfluorohexane may be manufactured as follows:

790 mg distearoylphosphatidylcholine (DSPC) and 8.1 mg stearylamine (SA) is weighed into a 250 ml round bottom flask and 50 ml chloroform is added. The sample is heated under hot tap water until a clear solution is obtained. The chloroform is removed by evaporation to dryness on a rotary evaporator at 350 mm Hg and 40° C., followed by further drying at 50 mm Hg in desiccator overnight. Thereafter, 160 ml water is added and the flask again placed on a rotary evaporator and the lipids rehydrated by full rotational speed and 80° C. water bath temperature for 25 minutes. The resulting lipid dispersion is transferred to a suitable vial and stored in refrigerator until use.

Emulsions are prepared by transferring aliquots of 1 ml of the cold lipid dispersion to 2 ml chromatography vials. Each of 6 vials are added 100 μl of the fluorocarbon oils as detailed above. The chromatography vials are shaken on a CapMix (Espe, GmbH) for 75 seconds. The resulting emulsions are washed three times by centrifugation and removal of infranatant followed by addition of equivalent volume of an aqueous 5 mM TRIS buffer. The vials are immediately cooled on ice, pooled and kept cold until use.

Coulter counter analysis is performed to determine the volume concentration and diameter of the microdroplets, and the emulsions are then be diluted with 5 mM TRIS buffer to a disperse phase concentration 4 μl microdroplets/ml.

Preparation of cluster compositions are performed by reconstituting Sonovue or Micromarker with 5 or 0.7 mL, respectively, of each of the C2 components described above.

### Results (Prospective):

Upon mixing of components C1 and C2, all six combinations are expected to comprise more than 10 million clusters per ml, with a mean diameter between 3 to 10 μm. The cluster compositions are expected to be useful in delivery of antimicrobial agents and for use in a method of treatment of an infection in accordance with the invention.

1. A method of treatment of an infection of a subject, comprising administering to the subject:
(a) a cluster composition comprising a suspension of clusters in an aqueous biocompatible medium, wherein the clusters have a mean diameter in the range of 1 to 10 μm, and a circularity <0.9, and comprising:
(i) a first component comprising a gas microbubble and a first stabiliser to stabilise the microbubble; and
(ii) a second component comprising a microdroplet comprising an oil phase and a second stabiliser to stabilise the microdroplet, wherein the oil comprises a diffusible component capable of diffusing into the gas microbubble so as to at least transiently increase the size thereof;
wherein the microbubbles and microdroplets of the first and second components have opposite surface charges and form the clusters via attractive electrostatic interactions; and
(b) at least one antimicrobial agent selected from the group consisting of an antibiotic, an antifungal, an antiviral and an antiparasitic as a separate composition from the cluster composition.

2. The method according to claim 1, comprising the steps of:
(i) administering the at least one antimicrobial agent to the subject;
(ii) administering the cluster composition to the subject; wherein the at least one antimicrobial agent is pre-, and/or co- and/or post administered to the cluster composition;
(iii) activating a phase shift of the diffusible component of the second component of the cluster composition from step (ii) by ultrasound insonation of a region of interest within the subject; and
(iv) facilitating extravasation of the at least one antimicrobial agent by further ultrasound insonation.

3. The method according to claim 2, wherein
for step (iii) the ultrasound insonation is at a first frequency of 1 to 10 MHz and with a first mechanical index of 0.1 to 0.4; and
for step (iv) the further ultrasound insonation is at a second frequency of 0.4 to 0.6 MHz and with a second mechanical index of 0.1 to 0.3.

4. The method according to claim 1, wherein the at least one antimicrobial agent is selected from the group consisting of an antibiotic agent and an antifungal agent.

5. The method according to claim 1, wherein the infection is a bacterial infection or a fungal infection.

6. The method according to claim 1, wherein the antimicrobial agent is selected from the group consisting of an antiviral agent and an antiparasitic agent.

7. The method according to claim 1, wherein the antimicrobial agent is selected from the Cmax dependent class of drugs.

8. The method according to claim 1, wherein the antimicrobial agent is selected from the % T>MIC dependent class of drugs.

9. The method according to claim 1, wherein the infection is a localized/focal infection.

10. The method according to claim 1, wherein the infection is at least one selected from the group consisting of bacterial meningitis, otitis media, an eye infection, sinusitis, an upper respiratory tract infection, pneumonia, a skin infection, gastritis, food poisoning, a urinary tract infection and a sexually transmitted disease.

11. The method according to claim 1, wherein the infection is related to an organ transplant.

12. The method according to claim 1, wherein the infection is at least one selected from the group consisting of endocarditis, a prosthetic joint infection (PJI), osteomyelitis, prostatitis, ventriculitis, brain abscesses, aspergilloma and acute bacterial cholangitis.

13. The method according to claim 1, wherein the clusters have a mean diameter in the range of 3-10 μm.

14. The method according to claim 1, wherein the cluster concentration of clusters in the size range of 1-10 μm is at least 25 million/ml.

15. The method according to claim 1, wherein a gas of the microbubbles comprises sulphur hexafluoride or a C3-6 perfluorocarbon or mixtures thereof.

16. The method according to claim 1, wherein an oil phase of the microdroplet comprises a partly or fully halogenated hydrocarbon or a mixture thereof.

17. The method according to claim 1, wherein the microbubble comprises a first stabilizer comprising a phospholipid, a protein, or a polymer optionally added a negatively charged surfactant, and the microdroplet comprises a second stabilizer comprising a phospholipid, protein, or a polymer optionally added a positively charged surfactant.

18. A method of delivering an antimicrobial agent to a subject with an infection, comprising the steps of:
(i) administering at least one antimicrobial agent selected from the group consisting of an antibiotic, an antifungal, an antiviral and an antiparasitic to the subject;
(ii) administering a cluster composition comprising a suspension of clusters in an aqueous biocompatible medium, wherein the clusters have a mean diameter in the range of 1 to 10 μm, and a circularity <0.9, and the cluster composition comprises:
(1) a first component comprising a gas microbubble and a first stabiliser to stabilise the microbubble; and
(2) a second component comprising a microdroplet comprising an oil phase and a second stabiliser to stabilise the microdroplet, wherein the oil comprises a diffusible component capable of diffusing into the gas microbubble so as to at least transiently increase the size thereof;
wherein the microbubbles and microdroplets of the first and second components have opposite surface charges and form the clusters via attractive electrostatic interactions, to the subject;
wherein the at least one antimicrobial agent is pre-, and/or co- and/or post administered to the cluster composition;
(iii) activating a phase shift of the diffusible component of the second component of the cluster composition from step (i) by ultrasound insonation of a region of interest within the subject; and
(iv) facilitating extravasation of the antimicrobial agent(s) administered in step (i) by further ultrasound insonation.

19. The method according to claim 18, wherein for step (iii) the ultrasound insonation is at a first frequency of 1 to 10 MHz and with a first mechanical index of 0.1 to 0.4; and
for step (iv) the further ultrasound insonation is at a second frequency of 0.4 to 0.6 MHz and with a second mechanical index of 0.1 to 0.3.

20. The method according to claim 10, wherein the infection is a skin infection or a urinary tract infection.

21. The method according to claim 13, wherein the clusters have a mean diameter in the range of 4-9 μm.
